(19) United States
(12) Patent Application Publication
(10) Pub. No.: US 2011/0129925 A1
(43) Pub. Date: Jun. 2, 2011

(54) ZEOLITE AND BONE MIMETIC ZEOLITE BASED COATINGS FOR BIOIMPLANTS

(76) Inventors: Yushan Yan, Riverside, CA (US); Rajwant Singh Bedi, Riverside, CA (US)

(21) Appl. No.: 12/997,849

(22) PCT Filed: Jun. 12, 2009
(86) PCT No.: PCT/US09/03546

§ 371 Date: Feb. 16, 2011

(30) Foreign Application Priority Data
Jun. 13, 2008 (US) 61061288

(51) Int. Cl. C12N 5/07; B32B 9/04; B32B 15/04; B05D 5/00; B05D 1/18

(52) U.S. Cl. 435/402; 428/446; 428/450; 427/2.29; 427/2.24; 428/448

(57) ABSTRACT

The disclosure provides biocompatible metal compositions, methods of making such compositions and uses thereof, including a method of synthesizing zeolite coatings. The disclosure further provides the zeolite-hydroxyapatite composite coatings and methods of making them, which includes forming a base zeolite layer, forming a hydroxyapatite layer on the base zeolite layer, and interlocking the hydroxyapatite layer with an outer zeolite layer. The composite can be formed on a metal substrate for bioimplants, such as titanium alloy and/or stainless steel, which is used for bioimplants.

### FIELD OF THE INVENTION

The invention relates to biocompatible coating compositions on metals, methods of making such compositions and uses thereof.

### BACKGROUND

World population is witnessing an unprecedented increase in elderly population proportions. By 2050, people over 60 years of age will make up 21 percent of the total population as compared to only 10 percent in 2000. With increasing elderly proportions, age-related orthopedic and dental problems will become more prevalent. Treating damaged bone and teeth with bioimplants has become a critical factor in improving the quality of life at an older age. Therefore, a longer bioimplant lifespan is highly desired to prevent recurring surgeries for patients. Implants are manufactured using expensive titanium alloys that possess suitable corrosion resistance and biocompatibility, but they must be replaced after 10-15 years of implantation. Over the past 40 years, only marginal improvements have been made to improve material properties of implants to match those of natural bone. To extend implant lifespan, several biocompatible coatings have been proposed that attempt to improve interfacial bond between the implant material and bone. While these coatings are biocompatible, many fail due to their inferior corrosion resistance, and a mismatch of elastic modulus with bone. Significant improvements in material properties such as corrosion resistance, modulus, and hydrophilicity are required to obtain an optimal biomaterial-bone interface for improving osteointegration for longer implant lifespan.

Titanium and its alloys, along with cobalt-chromium alloys, have traditionally been used in orthopedic and dental implants due to their high corrosion resistance and high biocompatibility. Recently, biocompatibility of titanium alloy Ti6Al4V (˜90% Ti, 6% Al, 4% V) has been questioned due to the release of harmful Al and V ions into the surrounding tissue. Specifically, vanadium ions have been shown to be cytotoxic, while titanium ions can cause neurological disorders. Even a highly passive titanium surface (with a protective TiO₂ layer) may allow release of ions into the surrounding tissue under corrosive and biologically active oral conditions. Release of metallic ions and particles diminishes the biocompatibility of titanium and its alloys over a long implant lifespan.

### SUMMARY

The invention comprises the use of high-silica zeolites as coatings for medical implants to improve corrosion resistance and biocompatibility. High-silica zeolites are nonporous and remarkably corrosion resistant in strong acid, base and pitting aggressive media (e.g., NaCl solution). Also provided is an in-situ crystallization coating deposition process useful for coating complex shapes and in confined spaces. The invention further comprises zeolite coatings which are functionalized with hydroxyapatite crystals to make them more biocompatible and mechanically compatible with bone.

Various (MFI (ZSM-5), BEA, MTW (ZSM-Twelve)) high-silica-zeolite coatings are all good corrosion resistant coatings. Zeolite coatings are barrier coatings that exhibit excellent adhesion to various metallic substrates (Al, Steel, Cu, Ni), and are known for their thermal, chemical and mechanical stability. As synthesized zeolite coatings are also impermeable to all gases, and do not react with any mineral acids but hydrofluoric acid. Zeolites can be used on any metal medical implant, including titanium, steel, aluminum, nickel or alloys and mixtures thereof. One simple pretreatment and a single zeolite formulation on all metal substrates to synthesize zeolites coatings is effective.

The disclosure demonstrates that high silica coatings can be synthesized by in-situ crystallization on titanium materials such as commercially pure titanium (cpTi) and Ti6Al4V, and show excellent corrosion resistance by DC polarization. Corrosion resistance of MFI coated titanium alloy was better than the uncoated titanium alloy, and did not deteriorate over time (see FIG. 4). Detailed preparation procedure for zeolite coatings and their corrosion resistance are provided. The zeolite coatings of the invention show a strong correlation between corrosion resistance and biocompatibility.

The invention further comprises the use of zeolite coatings which are functionalized with hydroxyapatite, a mineral found in bone. In accordance with an exemplary embodiment, a novel zeolite-hydroxyapatite composite is adhered to the base zeolite coating to enhance the biocompatibility of metallic implants by improving corrosion resistance and hydrophilicity while minimizing modulus mismatch with bone. It can be appreciated that zeolite-hydroxyapatite coating possesses bone-like mechanical properties, which prevent implant loosening and enhance osteointegration. In addition, the coating can prevent the underlying metallic implant from corroding and causing tissue damage and failure of implant. It can also be appreciated that a zeolite-hydroxypatite coating can reduce the need for recurring surgeries for implant replacement and improve the quality of life for millions of people with orthopedic and dental implants.

Hydroxyapatite is widely used in bioactive glass and bioceramics to form biocompatible coatings on metallic implants. It is inert in nature, and has a chemical composition similar to that of bone. In addition, hydroxyapatite is secreted by bone cells to form the extracellular matrix, which turns into solid bone. Hence, osteoblasts have favorable interactions with hydroxyapatite allowing them to adhere well to the bioceramic coating on the metallic implant. Currently, physical vapor deposition, sintering, and electrochemical growth of hydroxyapatite are the methods of choice for producing hydroxyapatite coatings on metallic implants. Although adhesion of cells to the hydroxyapatite coatings has been shown to be good, hydroxyapatite coatings have poor adhesion to the metallic substrate and are vulnerable to delamination resulting in failure of the implant. This can further lead to the corrosion of the implant and the release of toxic ions and wear particles into the neighboring tissue causing cell death by necrosis. In accordance with an exemplary embodiment, a zeolite-hydroxyapatite composite coating is disclosed, which enhances coating-substrate adhesion, and further mimic bone properties.

Zeolites are aluminosilicates with porous microcrystalline structure; their porosity has been essential for several industrial applications such as separations and catalysis. Various types of zeolite structures exist in nature, and are non-toxic to living beings, but commercial applications revolve around synthetic zeolites. Their non-toxicity has recently been exploited by researchers as drug delivery and MRI contrast agents. Although most applications of zeolites make use of their porous structure and powder form, their corrosion resistance properties stem from direct synthesis of zeolite coatings on metallic surfaces with occluded pores.

In accordance with an exemplary embodiment, silica zeolite (HSZ) MFI (ZSM-5) coatings are disclosed for corrosion protection of Ti6Al4V implant surfaces to prevent the release of neurodegenerative Al and cytotoxic V ions. MFI zeolite coatings have been shown to have high corrosion resistance, and excellent adhesion to various metallic substrates including titanium alloys. Zeolite MFI coatings also have high biocompatibility as compared with glass and bare Ti6Al4V alloys. In addition, MFI surfaces have superior osteoconductive and osteoinductive properties than bare Ti6Al4V, and to enhance these properties hydroxyapatite can be incorporated into the MFI coating. Presence of hydroxyapatite on a surface stimulates differentiation of human fetal osteoblasts (hFOBs) into adult osteoblasts, and enhances bone proliferation due to enhanced osteoblast activity.

In accordance with an exemplary embodiment, it would be desirable to take advantage of both of these qualities by forming a base MFI layer on the substrate, followed by a hydroxyapatite layer on the MFI layer by dip coating, and a subsequent short MFI synthesis to lock-in the hydroxyapatite crystals (see FIG. 14). Characterization of the coating properties was performed to study the coating topography, composition, corrosion resistance, and hydrophilicity. Mechanical properties of the coating were determined and then compared to those of natural bone.

In accordance with an exemplary embodiment, a method of synthesizing a zeolite-hydroxyapatite composite comprises: forming a base zeolite layer; forming a hydroxyapatite layer on the base zeolite layer; and interlocking the hydroxyapatite layer with an outer zeolite layer.

In accordance with an exemplary embodiment, a zeolite-hydroxyapatite composite comprises: a base zeolite layer; a hydroxyapatite layer on the base zeolite layer; and an outer zeolite layer on the hydroxyapatite layer.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

FIGS. 1A-1D are (A) a scanning electron microscope (SEM) micrograph of cpTi; (B) an EDS analysis of cpTi; (C) a SEM micrograph of MFI coating on cpTi; and (D) a EDS analysis of MFI coating on cpTi.

FIGS. 2A-2D are (A) a SEM micrograph of Ti6Al4V surface; (B) an EDS analysis of Ti6Al4V; (C) a SEM micrograph of MFI coating on Ti6Al4V surface; and (D) an EDS analysis of MFI coating on Ti6Al4V.

FIGS. 3A-3D are (A) a SEM micrograph of the zeolite-Ti6Al4V interface; (B) a SEM micrograph overlaid with EDS linescan indicating incorporation of Ti into the zeolite framework; (C) a SEM micrograph of the zeolite-cpTi interface; and (D) a SEM micrograph overlaid with EDS linescan indicating incorporation of Ti into the zeolite framework from cpTi.

FIG. 4 is a comparison of corrosion resistance of MFI coated and bare Ti6Al4V in (a) 0.856M NaCl solution at pH of 7.0, and (b) 0.856M NaCl solution at pH of 1.0, and wherein the legend shows immersion time from 5 minutes (5 m) to 7 days (7 d), with asterisk (*) indicating MFI coating applied to the metal, represented by solid lines.

FIGS. 5A-5D are SEM micrographs of mouse embryonic stem cells on a fibroblast monolayer on (A & B) MFI coating on Ti6Al4V, and (C & D) glass coverslips.

FIGS. 6A-6H are (A) a SEM micrograph of bare Ti6Al4V; (B) an EDS scan showing composition of the alloy; and SEM micrographs of osteoblasts cultured on bare Ti6Al4V for (C) 24 hours, (D) 4 days, (E) 7 days, (F) 14 days, (G) 21 days, and (H) 30 days.

FIGS. 7A-7H is (A) a SEM micrograph of MFI-coated Ti6Al4V; (B) EDS scan showing composition of the zeolite coating; and SEM micrographs of osteoblasts cultured on MFI-coated Ti6Al4V for (C) 24 hours, (D) 4 days, (E) 7 days, (F) 14 days, (G) 21 days, and (H) 30 days.

FIGS. 8A and 8B are DC Polarization curves comparing the corrosion resistance of bare (A) and MFI-coated (B) Ti6Al4V.

FIG. 9 is a Trypan blue cell proliferation assays comparing growth of hFOBs on bare and MFI-coated Ti6Al4V over 7 days in DMEM:F-12 complete growth medium, wherein the Mean±standard deviation values are reported with n=3, and wherein [*] indicates the values are significantly higher on MFI than on Ti6Al4V with p<0.05.

FIG. 10 is a Vonkossa staining of osteoblast cultures on bare and MFI-coated Ti6Al4V over one month period, and wherein images are shown at 5× (5 times) magnification.

FIG. 11 is a concentration profiles of total RNA extracted from osteoblasts cultured on bare and MFI-coated Ti6Al4V substrates for one month.

FIG. 12 is a gel electrophoresis confirmation of expression of osteoblast genes tested.

FIGS. 13A and 13B are charts showing number of cycles required to reach threshold for amplification of test genes with respect to GAPDH for (A) bare Ti6Al4V and (B) MFI-coated Ti6Al4V, and wherein the numbers reported are mean±standard deviation of triplicates, and [*] indicates that values are significantly higher gene expression on MFI than Ti6Al4V with a p<0.05.

FIG. 14 is a schematic of zeolite-hydroxyapatite composite coating, wherein the zeolite MFI forms an adhesive corrosion resistant layer on the metal surface, and the hydroxyapatite nanocrystals are deposited on the base layer, and locked in with another MFI synthesis in accordance with an exemplary embodiment.

FIGS. 15A-15D are SEM images of (A) a surface of MFI-HA coating on Ti6Al4V substrate, ((B) and (C)) surface intergrowth of MFI-HA after 4 hour short MFI synthesis, and (D) slight recrystallization of hydroxyapatite observed after 4 hour short MFI synthesis in accordance with an exemplary embodiment.

FIG. 15E is an EDS analysis showing the presence of Ca and P ions after 4 hour short MFI synthesis in accordance with an exemplary embodiment.

FIG. 15F is a chart showing contact angle measurements showing an increase in hydrophilicity of MFI-HA coating as compared to MFI coated and bare Ti6Al4V in accordance with another exemplary embodiment.

FIGS. 16A-16D are XRD (X-ray diffraction) patterns indicating incorporation of MFI into the composite coating and no loss of hydroxyapatite crystallinity after 4 hour short MFI synthesis in accordance with an exemplary embodiment.

FIGS. 17A-17F are comparison of corrosion resistance of bare (▴) and MFI-HA-coated () Ti6Al4V (A-C) and SS316L (D-F) in 0.856 M NaCl solution, 1×PBS+1 mg/ml BSA solution, and 50:50 DMEM/F-12 solution respectively.

FIGS. 18A and 18B are images of material properties of MFI-HA coatings such as (A) modulus and (B) hardness, obtained experimentally by nano-indentation, are compared to nano-indentation values found in literature for SS316L, Ti6Al4V, MFI coatings, Trabecular bone, Cortical 1 bone vertical testing) and Cortical 2 bone (horizontal testing).

FIG. 19 is a chart showing cell proliferation assay results on bare and MFI-HA coated Ti6Al4V and SS316L substrates over a 7 day period.

FIG. 20 are images of RNA extracted from hFOBs cultured on MFI-HA coated and bare Ti6Al4V and SS316L substrates over 30 days of culture.

FIG. 21 is an RT-qPCR analysis of hFOB gene expression over 14 days of culture on bare and MFI-HA coated Ti6Al4V and SS316L, wherein (*) indicates a significant increase in gene expression on MFI-HA coated Ti6Al4V and SS316L versus their corresponding bare substrates with a p<0.05, and the numbers reported are mean±standard deviation of triplicates; and lower values indicate fewer number of PCR cycles were needed to reach a specified expression level with respect to GAPDH, therefore they represent higher gene expression.

### DETAILED DESCRIPTION

As used herein and in the appended claims, the singular forms “a,” “and,” and “the” include plural referents unless the context clearly dictates otherwise. Thus, for example, reference to “an alloy” includes a plurality of such alloys and reference to “the material” includes reference to one or more materials, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Any publications discussed above and throughout the text are provided solely for their disclosure prior to the filing date of the present application. Nothing herein is to be construed as an admission that the inventors are not entitled to antedate such disclosure by virtue of prior disclosure.

Titanium and its alloys are overtaking other metallic compounds, such as stainless steel and cobalt chromium alloys, for use in biomedical implants where high corrosion resistance, high mechanical strength and biocompatibility are required. Since the early stages of biomaterials research, criteria for biocompatibility states that materials be inert and non-toxic. Although commercially pure titanium (cpTi) and Ti6Al4V (approximately 5.5 to 6.75% Al and approximately 3.5 to 4.5% V) are regarded as biocompatible, and thus chosen for dental and orthopedic implants, the release of metal ions and wear particles from these alloys after electrochemical dissolution appear to be one of the causes that result in poor osteointegration of titanium prosthesis. In addition, vanadium ions have been shown to be cytotoxic, while titanium ions can cause neurological disorders thus presenting a danger to human health. Even a highly passive titanium surface (with a protective TiO₂ layer) may allow release of ions into the surrounding tissue under corrosive and biologically active oral conditions. Thus, release of metallic ions and particles diminishes the biocompatibility of titanium and its alloys over an implant's lifespan.

Typically, these plates are used as short term implants (6 months), and after use analysis has shown the presence of Al and V ions in tissue surrounding these plates. To date various ceramic coatings have been applied to reduce or prevent the release of harmful ions from the dissolution of metal implants. The disclosure provides a zeolite-based coating that prevents electrochemical dissolution of the underlying metal, thus reducing the release of harmful ions into the surrounding tissue.

To date, researchers are not aware of any published attempts made to synthesize zeolite coatings on titanium and its alloys. This work exploits the biocompatibility and corrosion resistance properties of zeolites and extends zeolite coatings as a suitable platform for dental and orthopedic implants. Titanium alloys were coated with zeolite coatings to prevent the corrosion of titanium alloys and release of harmful ions. Zeolite coated titanium alloys have, at least, the following advantages compared with standard uncoated titanium alloys used in implants: (1) Uniform coatings can be obtained on commercially pure titanium (cpTi) and Ti6Al4V (˜90% Ti, 6% Al, and 4% V); (2) Excellent adhesion of zeolite coating to titanium alloys is observed; (3) Coatings are highly corrosion resistant in aggressive pitting (acidified and non-acidified) media-better than bare Ti6Al4V and do not lose their properties over time. Coatings prevent the electrochemical dissolution and release of toxic Al and V ions from the alloy; (4) Coatings are biocompatible—no cytotoxic effects to fibroblasts and stem cells; (5) Improved cell adhesion to zeolite coatings over glass substrate; and (6) zeolite coatings present a 3-D surface for cell growth, which increases cell proliferation as compared to 2-D flat surfaces of glass.

In accordance with an exemplary embodiment, the disclosure provides a method of making high-titanium zeolite coatings for titanium or titanium alloy substrates, as well as other corrodible metals.

In accordance with an exemplary embodiment, the embodiments of the disclosure are directed towards generating high titanium zeolite coatings on titanium and its alloys. In one embodiment, a high titanium zeolite is one where the zeolite has a silicon: titanium ratio range of less than 5. The embodiments of the disclosure describe such coatings and their application process. In accordance with a further embodiment of the disclosure, the coated titanium or titanium alloy substrate can have three layers (base, middle and top), each layer having a distinct synthesis process. Many zeolite molecular sieve compositions could be used for each of the layers. The base zeolite layer, directly in contact with the titanium substrate, is typically of pure or high silica zeolite such as Silicalite-1 or ZSM-5. The method for the formation of the base layer is a one-step in-situ crystallization at low temperature using synthesis solutions of mild or neutral pH. Pure or high silica zeolites have higher chemical, thermal and mechanical stability than their high titanium counterparts. The high silica zeolite base layer confers corrosion protection to the titanium substrate that will be helpful in protecting the titanium in the severely corrosive synthesis solution of high titanium zeolites.

In one embodiment, the zeolite coating can be comprised of various layers, including high titanium zeolites and intermediate layers to adhere high silica and high titanium layers. In such embodiments, the top layer is formed by seeded growth. The two-layer zeolite coated substrate described above is seeded with high titanium zeolite crystals followed by a short synthesis in a high titanium synthesis solution. The synthesis of this top layer may be repeated several times to achieve a desired thickness.

The embodiments of the disclosure are useful for generating hydrophilic, high titanium zeolite coatings on titanium and its alloys. Zeolites, especially pure and high silica zeolites are known for their thermal and chemical stability and mechanical strength. Several high silica zeolite coatings such as ZSM-5 are corrosion resistant and have superior performance to chromate conversion and anodization coatings. These corrosion resistant zeolite coatings can be universally applied to many metal types. The metals demonstrated have included various steels (including SS316L), titanium, and titanium alloys, including the 2000, 5000, 6000, and 7000 series aluminum alloys. The coating process is in-situ crystallization, which is capable of coating surfaces of complex shape and in confined spaces. Once synthesized the high silica zeolite coatings afford corrosive protection of the coated substrate in strongly corrosive media, including extremely acidic and basic environments.

The crystalline structure of the high silica (i.e., the base layer) and high titanium zeolites (i.e., the top layer) are vastly different. To ensure strong adhesion between the two different coating types a mixed zeolite layer consisting of both types of zeolites is formed. This middle layer serves as an anchoring bridge between the two types of zeolite coatings. The high silica zeolite coated titanium substrate is coated with either a mixture of high silica and high titanium zeolite seed crystals or solely with high titanium zeolite seed crystals. Many seeding permutations may be used. In one arrangement, the seed layer is a mixture of the two seed types with the high titanium seed making up at least 50% of the seed mixture. The mixed seed layer undergoes a short, (e.g., one-hour) synthesis in high silica zeolite synthesis solution. The short synthesis ensures that the high titanium zeolite crystals are anchored and the high silica layer is thin so as to leave exposed the high titanium zeolite crystals. As used herein, a short synthesis is a time significantly less than the time required for the high silica base layer and a thin layer refers to the thickness of the high silica component of the mixed zeolite middle layer. Longer synthesis times would result in the high silica zeolite component masking the high titanium zeolite seed crystals present in the layer.

As described above, the top layer is a high titanium zeolite layer. Such zeolite coatings include zeolite X, zeolite Y, zeolite A, and others. In one embodiment, the top layer's coating process is also a seeded growth. Substrates with the middle and base (i.e., two layer coating structure) zeolite layers are initially seeded with nanometer-sized or micrometer-sized high titanium zeolite crystals followed by synthesis in a high titanium zeolite synthesis solution. The top layer synthesis can be repeated several (e.g., three) times to optimize coating thickness.

In addition to the embodiments described above for forming high titanium zeolite coatings on titanium and titanium alloy substrates, the following alternate embodiments may also be used to form high titanium zeolite coatings on an titanium, titanium alloy, and other corrodible metal substrates.

In an alternative embodiment, as an alternative to seeded growth, the top (the high titanium zeolite) layer may be formed by an in-situ crystallization process. For example, the inventors herein have successfully generated a high titanium zeolite, zeolite A.

In accordance with another exemplary embodiment, a method of synthesizing a zeolite-hydroxyapatite composite comprises the steps of forming a base zeolite layer; forming a hydroxyapatite layer on the base zeolite layer; and interlocking the hydroxyapatite layer with an outer zeolite layer.

In accordance with an exemplary embodiment, the zeolites as described herein are preferably an aluminosilicate, and more preferably a high-silica or pure-silica zeolite having a Si/Al₂O₃ (Silica/Alumina) ratio of approximately at least 5:1, and more preferably a Si/Al₂O₃ ratio of approximately 50:1 or higher for the base (or bottom) zeolite layer. However, it can be appreciated that other silica zeolites can also be used, such as those disclosed in Y. Yan, H. Wang 2004, [Invited Review], “Nanostructured Zeolite Films” in Encyclopedia of Nanoscience and Nanotechnology, Edited by H. S. Nalwa, American Scientific Publishers, Volume 7, p. 763-781; Cheng X, Wang Z, Yan Y. Corrosion-Resistant Zeolite Coatings by In Situ Crystallization. Electrochemical and Solid-State Letters 2001; 4(5):B23-B26; U.S. Pat. No. 6,521,198 entitled “Metal surfaces coated with molecular sieve for corrosion resistance” issued to Yushan Yan, et al.; and U.S. Provisional Patent Application Ser. No. 61/103,448 entitled “Ambient Pressure Synthesis of Zeolite Films and their application as Corrosion Resistant Coatings” filed by Yushan Yan, et al, each of which is incorporated herein in their entirety.

It can be appreciated that in accordance with an exemplary embodiment of the zeolite-hydroxyapatite composite, the outer (or top) zeolite layer does not have to match the base (or bottom) zeolite layer. As described herein, the outer (or top) layer locks in the hydroxyapatite crystals, and therefore the top zeolite layer can be a layer that adheres properly to the base (or bottom) MFI layer and incorporates the hydroxyapatite crystals. Thus, in accordance with an exemplary embodiment, the outer (or top) layer can be a high silica MFI or other high silica zeolite.

In accordance with an exemplary embodiment, the bottom zeolite layer should be sufficiently thick to provide excellent corrosion resistance within and also be formed reasonably quickly for fast processing. Typically, a 24 hour synthesis is needed for the deposition of 7-8 micrometer thick coating, which is highly corrosion resistant. The outer (or top) layer on the other hand can have variable thickness as long as the hydroxyapatite (HA) crystals are still visible on the surface for preserving bioactivity of the coating. In accordance with an exemplary embodiment, the outer (or top) layer thickness can range from approximately 10 to 100 micrometers. For example, a four hour synthesis can produce a 10 micron thick coating with hydroxyapatite (HA) crystals on the surface of the substrate. Accordingly, it is desirable that the hydroxyapatite (HA) crystals show good adhesion to the base layer and do not delaminate.

In accordance with another exemplary embodiment, the zeolite coatings can be prepared by in-situ crystallization and/or a spin-on technique. For example, a zeolite synthesis composition is first formed by combining a silica source with an organic zeolite-forming structure-directing agent (“SDA”). The silica source is preferably an organic silicate, most preferably a C₁-C₂ orthosilicate such as tetraethyl orthosilicate (TEOS) or tetramethyl orthosilicate (TMOS). However, inorganic silica sources such as fumed silica, silica gel or colloidal silica can also be used. The zeolite-forming structure-directing agent is typically an organic hydroxide, preferably a quaternary ammonium hydroxide such as tetrapropylammonium hydroxide (TPAOH), tetraethylammonium hydroxide (TEAOH), triethyl-n-propyl ammonium hydroxide, benzyltrimethylammonium hydroxide, and the like. The resulting synthesis composition contains ethanol, if TEOS is used as the silica source, or methanol, if TMOS is used.

In the in-situ crystallization process in accordance with an exemplary embodiment, the molar composition of the synthesis composition is xSDA/1 silica source/yH₂O. X can range from about 0.2 to about 0.6, preferably from about 0.2 to about 0.45, and most preferably 0.32. Y can range from about 100 to 200, preferably from about 140 to about 180 and is most preferably 165.

In the in-situ crystallization process, in general, the metal substrate (or substrate) to be coated is brought into contact with the synthesis composition inside a reaction vessel such as an autoclave. The vessel is then sealed and placed in an oven. If a convection oven is used, heating is generally conducted at a temperature of from about 120° C. to about 190° C., preferably from about 160° C. to about 170° C. and most preferably about 165° C. A microwave oven can also be used, in which case the power level is preferably high and the time is from 5 to 30 minutes, preferably 10 to 25 minutes, and most preferably about 10 minutes. The drying step is preferably followed by heating conducted at temperatures of from about 350° C. to about 550° C., preferably from about 400° C. to about 500° C. This heating step, usually referred to as a calcination step, accomplishes removal of the SDA from the coating and can improve the coating's adhesion and strength.

The zeolite coatings produced by this process are generally hydrophobic; consequently, their properties are relatively uninfluenced by moisture. If desired, hydrophobicity may be increased further by removal of surface hydroxyl groups by silylation, for instance, with chlorotrimethylsilane, as described below, by high temperature oxidation, or by other techniques known in the art for this purpose.

In accordance with another exemplary embodiment, a zeolite synthesis composition containing an SDA, a silica source (as described above) and water is prepared. The molar composition of the synthesis composition is x₁ SDA/1 silica source/y₁ H₂O. X₁ can range from about 0.2 to about 0.5, preferably from about 0.3 to about 0.4, and most preferably 0.36. Y₁ can range from about 5 to about 30, preferably from about 10 to about 20, and most preferably 14.29.

In conducting this process, the above synthesis composition is prepared. Then the composition is loaded in a vessel, which is sealed, and the composition is heated to a temperature of from 40 to 100° C., preferably 60 to 90° C., and most preferably 80° C. The heating time is from 1 day to 7 days, preferably 2 to 4 days, and most preferably 3 days. A suspension of zeolite crystals is produced.

In accordance with an exemplary embodiment, the suspension is then centrifuged or otherwise treated to recover nanocrystals (i.e., nanometer-sized crystals). The crystals are then re-dispersed in ethanol or another appropriate dispersant, and are placed on a metal substrate that is situated on a spin coater. Spin coating is then conducted as known in the art by rotating the substrate at high speeds such that a highly uniform coating is obtained on the substrate. Preferably the coating is subjected to a brief drying step (e.g. about 10-12 minutes at 100° C.). Finally the coating is subjected to a heating (“calcination”) step. This step is conducted at temperatures of from about 350° C. to about 550° C., preferably from about 400° C. to about 500° C. It accomplishes removal of the SDA from the coating and can improve the coating's adhesion and strength.

In accordance with another exemplary embodiment of the spin-on process, involving a spin-on of as-synthesized nanoparticle suspension, methanol or ethanol is included in the initial synthesis composition. If a lower alkyl orthosilicate is used as the silica source, methanol or ethanol is chosen as corresponding to the alkyl groups. This is in addition to any amount formed by the hydrolysis of the organic silica source. If an organic silica source is used, either methanol or ethanol may be used. The molar composition of the synthesis composition if x₂ SDA/1TEOS/z₂ EtOH(or MeOH)/y₂ H₂O. X₂ can range from about 0.2 to about 0.5, preferably from about 0.3 to about 0.4, most preferably 0.36. Y₂ can range from about 10 to 20, preferably from about 12 to about 18, most preferably 14.29. Z₂ can range from about 1 to about 10, preferably from about 2 to about 6, most preferably 4.0.

It can be appreciated that in this embodiment it is not necessary to collect and then re-disperse the zeolite nanocrystals, and the suspension (without re-dispersion) is subjected to spin coating as described above, followed by optional drying and then heating or calcination.

In accordance with another exemplary embodiment, when the zeolite precursor or synthesis composition is formed using excess ethanol or methanol, as above, the resulting suspension may also be used to produce silica zeolite coatings having surface patterns. Ethanol is preferred for this process. However, instead of in-situ crystallization or spin coating, the suspension is simply deposited on an appropriate substrate and allowed to dry at ambient temperatures. Surface patterns are believed to form spontaneously as a result of convection due to the evaporation of the excess ethanol. Eventually the suspension dries completely, and the zeolite nanoparticles become locked into solid patterns. The use of ethanol as opposed to another alcohol such as propanol, the presence of excess ethanol in the system (as opposed to only the amount generated between the template and the silica source), and the crystal size in the suspension, are important factors in the production of surface-patterned silica zeolite films by this process. Preferably, the suspension contains crystals of about 25-50 nm diameter, as well as smaller nanoslabs and nanoslab aggregates.

The properties of zeolite coatings produced by spin-coating can be varied in several ways. The zeolite coating thickness can be increased, if desired, by conducting the spin-on process two or more times, with additional material added on each occasion. If the zeolite coating is produced by the first embodiment of the spin-on process, that is, one in which crystals are redispersed before the spin-on is conducted, the adhesion of the film to the substrate may not be strong enough to withstand treatments such as mechanical polishing. If that is the case, the calcined film can be treated by exposing it to microwaves in the presence of additional zeolite synthesis or precursor solution, or by heating it with additional zeolite precursor solution in a convection oven or similar equipment. This produces a secondary growth of zeolite on the substrate, but if the treatment is kept reasonably brief (perhaps less than 15 minutes for microwaving), the film thickness does not significantly increase.

The films or coatings produced by the spin-on processes also are generally hydrophilic. To minimize or prevent adverse affects due to moisture, these films or coatings can be made substantially hydrophobic by treatments to remove surface hydroxyl groups, such as by silylation (with chlorotrimethylsilane, for example), high temperature oxidation, or other techniques known in the art for this purpose.

The embodiments of the disclosure enable the treatment of any metal surface, that is otherwise susceptible to corrosion upon exposure to a highly acidic or alkaline solution, to render it effective for adsorption of water and other molecules. Exemplary metals include titanium-containing metals, titanium alloys and titanium and steels (especially SS316L, which is used to form bioimplants). The following additional examples are offered for purposes of illustration, and are not intended to limit the invention.

### EXAMPLES

Coating Solution Formulation.

High-silica zeolite (HSZ) MFI coatings were prepared by an in-situ hydrothermal crystallization method. First, a clear synthesis solution with molar composition 0.16TPAOH:0.64NaOH:TEOS:92H₂O:0.0018Al (weight compositions: 17.03 g TPAOH, 5.36 g NaOH, 43.60 g TEOS, 336.00 g H₂O, 0.0105 g Al) was prepared by dissolving titanium powder (200 mesh, 99.95+wt %, Aldrich) in sodium hydroxide (99.99 wt %, Aldrich) and de-ionized (DI) H₂O followed by drop-wise addition of tetrapropylammonium hydroxide (TPAOH, 40 wt %, aqueous solution, SACHEM, Austin, Tex.) and tetraethylorthosilicate (TEOS, 98 wt %, Sigma-Aldrich, St. Louis, Mo.) under stirring. The clear solution was aged at room temperature for about 4 hours under stirring before use.

Substrate Pretreatment.

Titanium alloys were commercially purchased. Commercially pure titanium (cpTi, 99.5%, 0.25 mm thick) was purchased from Alfa Aesar and high strength titanium (Ti6Al4V, 6% Al, 4% V, 0.41 mm thick) was purchased from McMaster Carr (Cleveland, Ohio). The substrates were sized to 15.25 cm×7.62 cm panels and cleaned at 70° C. for 1 hour in an Alconox® detergent solution prepared with 3.0 grams Alconox® (Sigma-Aldrich, St. Louis, Mo.) in 400 mL deionized (DI) H₂O. The substrates were then rinsed under DI H₂O with mild rubbing. Substrates were dried with compressed air and kept at ambient conditions for less than 1 hour before immersion in HSZ-MFI synthesis solution.

HSZ-MFI Coating Deposition.

A 2 L Teflon-lined Parr (Moline, Ill.) autoclave was used as the synthesis vessel and the substrate was suspended vertically inside the synthesis solution using a Teflon® holder and steel wire. Crystallization was carried out in a convection oven at 175° C. for 24 hours. The autoclave was then removed and quenched with tap water. The coated sample was rinsed with DI H2O and dried in ambient room air for at least 12 hours before characterization. Large coated substrates were then cut into 1.5 cm×1.5 cm for cell culture, and into 2.5 cm×3.8 cm coupons for DC polarization testing. Before immersing the coupons into corrosive media, the sides of the coupons were covered with 5-minute (Grainger) epoxy to prevent any release of ions from the edges.

Characterization using Scanning Electron Microscope (SEM), and Focused Ion Beam (FIB) Milling and Energy Dispersive X-ray Spectroscopy (EDS).

Scanning electron microscope (SEM) micrographs were obtained on a Philips XL30-FEG scanning electron microscope operated between 5 and 20 kV. An Au/Pd coating was applied to MFI coated cpTi and Ti6Al4V samples by sputtering for 20 seconds for SEM imaging. A Focused Ion Beam (FIB, Leo XB1540), equipped with a Gallium ion gun, was used to mill a 15 μm deep trench in the zeolite-titanium system. Incorporation of titanium in zeolite structure was determined by semi-quantitative energy dispersive X-ray spectroscopy (EDS), performed using the EDAX (Mahwah, N.J.) analytical system attached to the FIB column.

Direct Current (DC) Polarization and Biocompatibility Testing.

Polarization testing was carried out with a Solartron potentiostat SI 1287 in a three-electrode configuration with the zeolite coated substrate as the working electrode, a platinum foil as the counter electrode, and an Ag/AgCl saturated KCl electrode as the reference electrode. The corrosive medium was either 0.856 M NaCl (pH=7.0) or 0.856 NaCl/HCl (pH=1.0) aqueous solution. The edges of the coated substrate were sealed with epoxy while exposing the coating surface on which corrosion resistance was to be measured. Zeolite coated samples were immersed in the corrosive medium for 0-7 days prior to the polarization test. Ambient temperature was maintained during all polarization tests. A scan rate of 1 mV/s was applied and all potentials were measured with respect to the reference electrode. After substrates were removed from the solution, the solution was tested for the presence of Al, Ti, and V ions using a Perkin Elmer 3000DV inductively coupled plasma optical emission spectrograph (ICP-OES).

Fibroblast and Stem Cell Culture & Specimen Preparation for Electron Microscopy.

A fibroblast cell (MEF cell line STO from ATCC) culture was prepared in Dulbecco's Modified Eagle's Medium (DMEM) with a 10% fetal bovine serum concentration. Cells were incubated at 10° C. for 24 hrs. Mouse embryonic stem cells (mESC line D3 from ATCC) were cultured on a 100% confluent fibroblast layer in a medium containing: 122 ml DMEM, 22 ml fetal bovine serum (inactive, 15%), 1.5 ml L-glutamine (4 mM), 1.5 ml sodium pyruvate (1%), 1.5 ml non-essential amino acids (1%), 750 μl penicillin/streptomycin (0.5%), 150 μl leukemia inhibitory factor (LIF, 1000 units/ml) to maintain pluripotency, and 15 μl 2-mercaptoethanol (0.1 mM). After 1 day of culture, cells were fixed in 2.5% glutaraldehyde in 0.1M sodium cacodylate for 1 hour, incubated in 1% osmium tetraoxide for another hour, and dehydrated in ethanol series. Samples were dried using Balzar's critical point dryer, and subsequently sputter coated with Au/Pd layer for SEM imaging. All zeolite surfaces were UV irradiated overnight before cell culture.

Film morphology and continuity were determined by X-ray diffraction, EDS (Energy Dispersive X-ray spectroscopy) and SEM (Scanning Electron Microscope). Corrosion protection was measured by DC polarization in several corrosive media after immersing the coated and uncoated Ti6Al4V coupons for 1, 2, 4, 7, and 30 days. After removing the coupons, media solutions were tested using ICP-OES (inductively Coupled Plasma Optical Emission Spectrometry) for the presence of Al, V, and Ti ions to determine whether any harmful ions are released.

Evaluation of corrosion resistance and biocompatibility of zeolite coatings on titanium alloys.

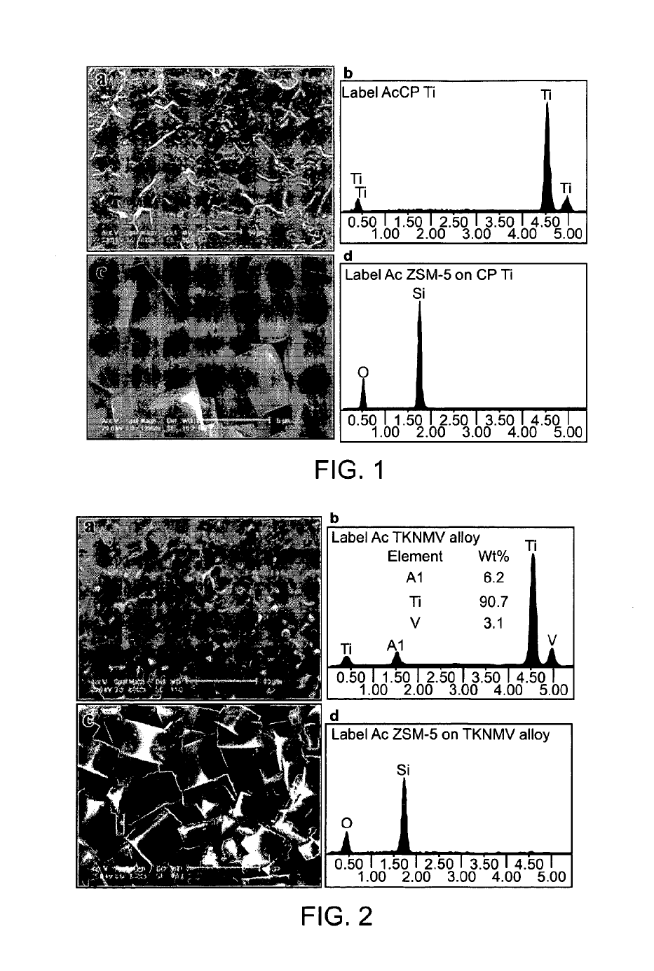

This work investigates zeolite MFI coatings for corrosion protection of Ti6Al4V in biomedical implants, and limiting the release of toxic Al and V ions into the tissue. Here, we have successfully synthesized MFI coatings on cpTi and Ti6Al4V with an approximate thickness of 8 μm, which can be varied by varying synthesis time. We obtained uniform MFI coatings on both cpTi and Ti6Al4V substrates using a one general solution formulation, and cleaning procedure. SEM micrographs show a highly crystalline surface without any microcracks or microporosity (FIGS. 1c & 2c) of the as-synthesized MFI coatings. EDS analyses confirmed the composition of the bare metal substrates to those presented by the manufacturer, and showed that MFI coatings on both substrates (FIGS. 1b, 1d & 2b, 2d) were comprised of Si and O.

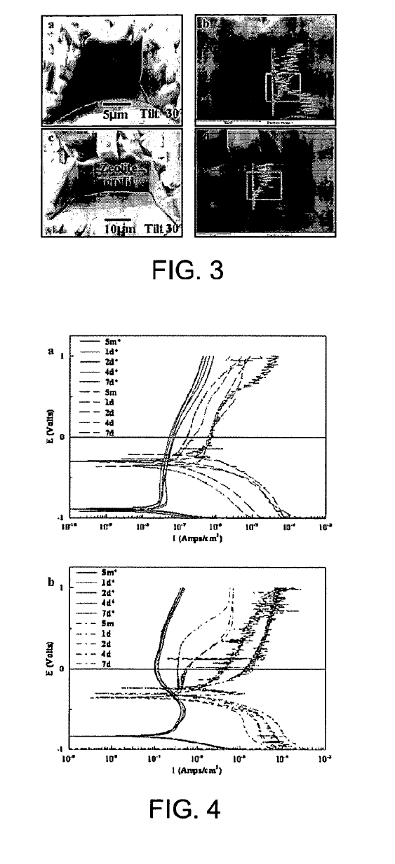

Focused Ion Beam milling was carried out to visualize the coating-substrate interface, and study the adhesion of the coating to the substrate. There is no observable gap between the metal surface and the MFI coating (FIGS. 3a and 3c). Some cracks are visible in the coating and the substrate, but they are away from the interface, or crossing the interface into the metal substrate. EDS linescan analysis performed on FIB milled interface of metal-zeolite interface revealed that Ti was incorporated into the zeolite framework up to approximately (˜) 4 μm deep in both cpTi and Ti6Al4V alloy (FIGS. 3b and 3d). This type of phenomenon has been previously reported by Beving et al. with Al substrates. Below the interface, a high intensity of Ti is observed, which starts to decrease at the interface and diminishes after penetrating about 4 μm into the MFI coating on both cpTi and Ti6Al4V substrates. At the atomic level, zeolites are constructed from TO₄ tetrahedra (T=tetrahedral atom, e.g., Si, Al); each apical oxygen atom is shared between two adjacent tetrahedra. Since Al and Si are tetrahedral atoms, incorporation of Al into the zeolite framework is understandable. It has also been shown that Ti octahedral can be interconnected with Si tetrahedral to form a network. Titanium silicates have been extensively studied by scientists, and are regarded as zeolite-like materials.

MFI coating on Ti substrates showed no microcracks indicating the presence of a uniform coating surface on the metal substrate. Therefore, the coating acted as a barrier for corrosion prevention of the metal substrate from aggressive pitting media. This hypothesis was confirmed by DC polarization results, which were carried out on bare Ti6Al4V and MFI coated Ti6Al4V surfaces after immersing the coupons for 5 min, 1, 2, 4, 7, and 30 days in both types of corrosive media: 0.856M NaCl solution at a neutral pH, and 0.856M NaCl/HCl solution at a pH of 1.0. A lower current density was observed in both cases for MFI coated samples as compared to bare Ti6Al4V samples (FIG. 4). MFI coated samples showed no change in current density over 7 days of immersion, while bare Ti6Al4V samples showed an increase in current density over time. A large increase in current density over time is apparent in the acidified corrosive media for the bare Ti6Al4V samples. Results indicated no change in corrosion current density after immersion of MFI coated Ti6Al4V samples in 0.856M NaCl at neutral pH and HCl acidified pH of 1.0 even after 30 days of immersion. Bending of polarization curves of MFI coating on Ti substrate in acidified media indicates the presence of a passivation layer thus referring to a higher corrosion resistance. In comparison, bare Ti6Al4V showed an increase in corrosion current density of several orders of magnitude in both the neutral and acidified corrosion media. Even with a highly passive TiO₂ surface layer, Ti6Al4V is prone to corrosion, and releases toxic ions.

MFI coatings can protect Ti6Al4V implants from corrosion in harsh acidic environment in the oral cavity, and thus reduce the release of toxic Al and V ions into the surrounding tissue. Solutions, in which bare and MFI coated Ti6Al4V samples were immersed, was tested for the presence of Al, V, and Ti ions using ICP-OES analysis (Table 1). ICP-OES analysis showed that while the release of metallic ions from Ti6Al4V was undetectable in 0.856M NaCl solution at neutral pH, acidified media accelerated the release of high levels of Al and V ions which can be cytotoxic to cells. Shi et al. demonstrated that V4+ is able to cause molecular oxygen-dependent hydroxylation and DNA strand breaks, while Cortizo et al. showed that vanadium compounds induce mitogenic effects correlated with morphological transformation on Swiss 3T3 fibroblasts. Here, the ICP-OES analysis detected no Al ions for bare Ti6Al4V in saline media at neutral pH, some Al was detected for MFI coated Ti6Al4V in the same media. The Al concentration detected was not significantly far off from the detection limit, and the Al concentration did not increase over time. Significant release of Al, Ti, and V ions was detected in acidified 0.856M NaCl media at pH of 1.0 in which bare Ti6Al4V was immersed, and concentrations increased with immersion time.

TABLE 1
Release of metal ions from MFI coated and bare Ti6Al4V surfaces after
immersion in neutral and acidified salt solution for 1 to 7 days.

| | Immersion | | MFI- | | MFI- |
|---|---|---|---|---|---|
| | time (days) | Bare^{[a]} | coated^{[a]} | Bare^{[b]} | coated^{[b]} |
| Mean | 1 | — | 10 | 251 | 14 |
| Al | 2 | — | 7 | 552 | 16 |
| Conc.^{[c]} | 4 | — | 6 | 529 | 17 |
| μg/L | 7 | — | 6 | 570 | 15 |
| Mean | 1 | — | — | 203 | — |
| Ti | 2 | — | — | 266 | — |
| Conc.^{[d]} | 4 | — | — | 681 | 6 |
| μg/L | 7 | — | — | 809 | 5 |
| Mean | 1 | — | — | 13 | — |
| V | 2 | — | — | 21 | — |
| Conc.^{[e]} | 4 | — | — | 37 | — |
| μg/L | 7 | — | — | 39 | — |

^{[a]}0.856M NaCl Media;
^{[b]}0.856M NaCl/HCl Media at pH = 1.0;
^{[c]}Det. Limit = 6 μg/L
^{[d]}Det. Limit = 2 μg/L; and
^{[e]}Det. Limit = 5 μg/L

Although MFI coated Ti6Al4V showed release of Al ions in neutral and acidified corrosion media, Al ion concentration did not increase after the first day. This can be explained by the release of any loosely attached Al to the zeolite surface, which was not fully incorporated into the zeolite structure before the synthesis was stopped. Although titanium is present in the zeolite coating, Beving et al. has previously reported by XPS that titanium concentration is graded, and a pure silica surface can be obtained. EDS analysis will still show the presence of Al in the coating, because the information obtained by EDS relies on the penetration of electrons into the sample. MFI coatings successfully prevented the release of cytotoxic V ions, while some Ti ions were observed after 4 days of immersion in the acidified media, and Ti concentration did not increasing after 7 days indicating a small amount of loosely incorporated Ti into the zeolite framework. The increase in the release of ion concentrations from Ti6Al4V in the acidified media matches the increase in corrosion current density of the samples with increasing time. Hence, the determination of biocompatibility of Ti6Al4V can potentially be achieved quickly by measuring the corrosion current density instead of performing long term immersion studies and analyzing release of metal ions. A higher current density will indicate poor corrosion resistance, and potential release of metallic ions from the substrate. While inertness can be determined in such a manner, determining overall biocompatibility requires cell culture studies.

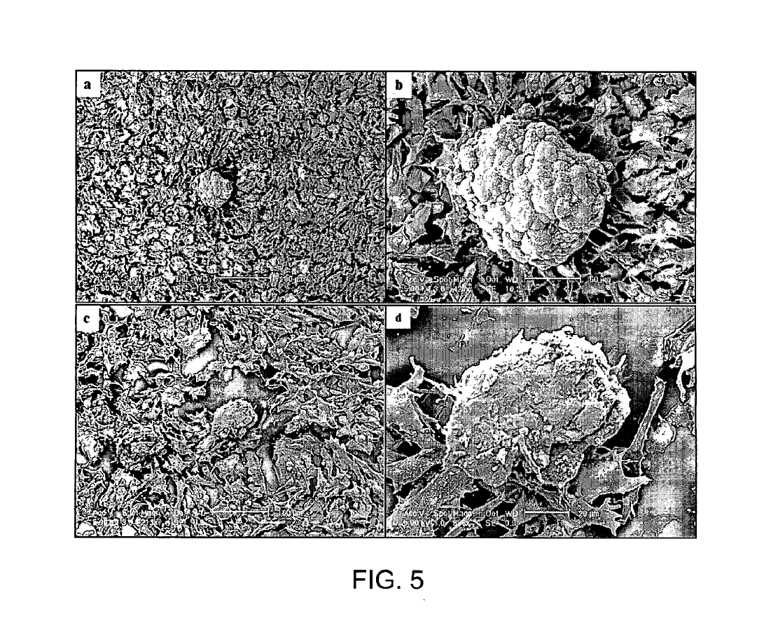

Fibroblast and stem cell culture data indicate that MFI coatings are biocompatible. To test the hypothesis that MFI coatings can be used in hard tissue regeneration, we cultured pluripotent mouse embryonic stem cells on MFI coated Ti6Al4V and glass coverslips, and studied the effect of the topography of the material surface on cell growth. A monolayer of mitotically inactivated fibroblasts was first seeded on the substrates to investigate cell adhesion, followed by culture of pluripotent mouse embryonic stem cells. We found that zeolites not only sustained, but favored the growth and attachment of both fibroblasts and embryonic stem cells (FIG. 5). Even though both surfaces—zeolites and control on glass—were composed mainly of silica, cells attached significantly better to the zeolite surface, indicating that the micro topography of zeolites offers higher proliferative properties. Round stem cell colonies were found on zeolite coatings, while a smaller number of flat cell colonies were observed on the glass surface (FIG. 5). Retaining their round shape could allow cells to function as if they were in their natural environment. Stem cell colonies on the zeolite surface were larger in size (approximately (˜) 150 μm long) versus the glass surface (approximately (˜) 80 μm long), indicating that zeolite surface could be better for cell proliferation. A greater number of elongations protruding out of the stem cell colony and attaching to the fibroblasts were found on zeolite compared with glass. In addition, a higher density of fibroblasts was seen on the zeolite surface. This indicates that zeolite coatings support proliferation of both fully differentiated (fibroblasts) and undifferentiated mitotically active mESC, with the potential for use in tissue regeneration. Higher cell densities, and smaller gaps between cells, were observed on zeolite than glass, indicating that a 3-D microstructure of zeolite crystals is favored for cell growth over the 2-D flat glassy surface. No cytotoxic effects on the stem cells or fibroblasts were observed on either the zeolite or the glassy surfaces.

Overall, results indicate that corrosion resistant zeolite coatings have the potential to be used as a biocompatible material for cell growth. MFI coatings exhibit higher corrosion resistance than Ti6Al4V, while providing a 3-D surface for cells growth. Zeolite coatings have good adhesion to titanium substrates and can potentially be used as biocompatible coatings to increase the lifespan of dental and orthopedic implants.

In accordance with an exemplary example, the corrosion resistance of zeolite MFI coatings were compared to bare Ti6Al4V alloy in PBS media, and the effect of zeolite 3-D microcrystalline topology on osteoblast proliferation was tested. A comparison of osteoblast proliferation and differentiation was made between bare Ti6Al4V and MFI coated Ti6Al4V surfaces. Gene expression of important osteoblast proliferation and differentiation markers ws quantified with PCR to determine whether hFOBs are performing their natural secretory functions and differentiate into adults osteoblasts. Functions of the osteoblast genes studied are listed in Table 2.

TABLE 2
Osteoblast genes tested with RT-QPCR and their functions.

| Gene | Function |
|---|---|
| GAPDH | Responsible for performing necessary metabolic and other |
| | cellular functions. Everything is normalized to GAPDH. |
| Collagen | Structural support and extracellular matrix formation. |
| T1A1[11] | |
| Osteopontin | Osteopontin binds to hydroxyapatite and is involved in |
| | anchoring osteoblast cells to bone cell matrix. |
| Osteocalcin | Important in bone metabolism and hydroxyapatite |
| | formation. |
| BMP2 | Induces osteogenic transformation. |
| RUNX2 | Principal osteogenic masterswitch for bone cell |
| | development and differentiation. |

It can be appreciated that zeolite coated titanium alloys have the following advantages compared with standard uncoated titanium alloys used in implants: (1) Coatings are highly corrosion resistant in aggressive pitting (acidified and non-acidified) media—better than bare Ti6Al4V—and do not loose their properties over time, and wherein coatings prevent the electrochemical dissolution and release of toxic Al and V ions from the alloy; (2) Coatings are biocompatible—no cytotoxic effects to osteoblasts; (3) MFI coatings present a 3-D surface for cell growth, which increases cell proliferation as compared to Ti6Al4V flat surfaces; (4) Osteoconductive and Osteoinductive properties of MFI coatings help osteoblasts proliferate faster, and mineralize hydroxyapatite faster than on bare Ti6Al4V.

Corrosion Resistance of MFI Coatings in Biological Media and Enhanced Functionality of Osteoblasts on MFI Coatings:

Substrate Preparation and Coating Synthesis

High strength titanium (Ti6Al4V, 6% Al, 4% V, 0.41 mm thick, McMaster Carr, Cleveland, Ohio) panels were cut into 1 inch×1.5 inch coupons for cell culture. Larger (3 inch×6 inch) panels were used to synthesize zeolite MFI (ZSM-5) coatings on the substrates via hydrothermal synthesis as previously described herein using the following solution formulation: 0.16TPAOH:0.64NaOH:TEOS:92H₂O:0.0018Al (weight compositions: 17.03 g TPAOH, 5.36 g NaOH, 43.60 g TEOS, 336.00 g H₂O, 0.0105 g Al). Briefly, Al powder (200 mesh, 99.95+ wt %, Aldrich, St. Louis, Mo.) was dissolved in NaOH (99.99 wt %, Aldrich, St. Louis, Mo.) and DI H₂O, and aged for 15 minutes. Dropwise addition of tetrapropylammonium hydroxide (TPAOH, 40 wt %, aqueous solution, SACHEM, Austin, Tex.) and tetraethylorthosilicate (TEOS, 98 wt %, Sigma-Aldrich, St. Louis, Mo.) was carried out, and solution was again aged for 4 hours to obtain a clear solution. Coating was deposited on Ti6Al4V by hydrothermal synthesis in a 2 L Teflon-lined Parr autoclave (Model #4622, Parr Instruments, Moline, Ill.) at 175° C. for 24 hours. Large panels were cut into 1 inch×1.5 inch coupons for cell culture.

Corrosion Resistance

Corrosion resistance of bare and MFI-coated Ti6Al4V was measured using DC Polarization method. Polarization tests were carried out in 1× phosphate buffer saline (PBS) solution using a Solartron (Farnborough, Hampshire, England) potentiostat SI 1287 in a three-electrode configuration with the substrate as the working electrode, a platinum foil as the counter electrode, and an Ag/AgCl saturated KCl electrode as the reference electrode. A scan rate of 1 mV/s was applied and all potentials were measured with respect to the reference electrode.

Osteoblast Cell Culture

Human fetal osteoblasts (hFOB, ATCC, Manassas, Va.) were cultured on bare Ti6Al4V and MFI-coated Ti6Al4V substrates in complete growth medium. The complete growth medium was prepared by mixing the following components: 500 mL DMEM:F-12 (Invitrogen, Carlsbad, Calif.) medium, 160 mg G428, and 55 mL fetal bovine serum (FBS). Cells were cultured in 5% CO₂ at a propagation temperature of 34° C. for 48 hours, after which cells were transferred to osteogenic mineralization media and cultured in mineralization medium at 39.5° C. The mineralization medium was prepared by adding 25 μL of 0.1 μM dexamethasone, 125 μL of 0.2 mM ascorbic acid and 2.5 mL of 10 mM glycerol-2-phosphate to complete growth medium to reach a final volume of 250 mL. Cells were cultured in the osteogenic mineralization medium for 30 days, and medium was replaced every 48 hours. Samples from cell cultures were collected for analysis after 1 day of incubation in complete medium, and on days 4, 7, 14, 21, and 30 while incubating in osteogenic mineralization medium.

Purification of Total RNA and Real-Time—Quantitative PCR (RT-QPCR)

Purification of total RNA from osteoblasts was carried out by using RNeasy Plus Mini Kit (Qiagen, Valencia, Calif.). Briefly, cells were lysed directly on the bare and MFI-coated Ti6Al4V, collected and homogenized using a 0.33 mm needle syringe. Subsequent washing with various buffers and centrifugations were carried out based on the protocol provided by Qiagen until the RNA was obtained. RNA concentration was measured using the Nanodrop-1000 spectrophotometer (Thermo Fisher Scientific, Wilmington, Del.).

QuantiTect® Reverse Transcription Kit (Qiagen, Valencia, Calif.) was used to transcribe RNA to cDNA. Quantity of RNA added to reverse transcription reaction varied depending on the RNA concentration within each sample. A total of 50 μg cDNA was obtained from each sample for amplification to establish a baseline.

Custom-made primers were ordered from Invitrogen (Carlsbad, Calif.), and the sequences used for amplification by PCR are shown in Table 2. RT-QPCR amplification was quantified using SYBR green ROX dye in the ABI PRISM® 7700 Real Time PCR System (Applied Biosciences, Foster City, Calif.). All PCR reactions were performed in triplicates and results are reported as mean±standard deviation. Following RT-QPCR amplification, agarose gel electrophoresis was performed in an ethydium bromide gel to determine the presence of each of these genes.

TABLE 3
Primer sequences used for amplification of RNA.

| | | Primer | Size | Accession |
|---|---|---|---|---|
| Gene | Direction | Sequence | (bp) | Number |
| GAPDH | Forward | GGCCTCCAAG | 147 | NM002046 |
| | | GAGTAAGACC | | |
| GAPDH | Reverse | AGGGGTCTAC | | |
| | | ATGGCAACTG | | |
| Collagen | Forward | ACGTCCTGGT | 172 | NM000088 |
| T1A1 | | GAAGTTGGTC | | |
| Collagen | Reverse | ACCAGGGAAG | | |
| T1A1 | | CCTCTCTCTC | | |
| Osteopontin | Forward | TTGCAGTGAT | 115 | NM001040058 |
| | | TTGCTTTTGC | | |
| Osteopontin | Reverse | GCCACAGCAT | | |
| | | CTGGGTATTT | | |
| Osteocalcin | Forward | GACTGTGACG | 119 | NM199173 |
| | | AGTTGGCTGA | | |
| Osteocalcin | Reverse | CTGGAGAGGA | | |
| | | GCAGAACTGG | | |
| BMP2 | Forward | TCAAGCCAAA | 103 | NM001200 |
| | | CACAAACAGC | | |
| BMP2 | Reverse | AGCCACAATC | | |
| | | CAGTCATTCC | | |
| RUNX2 | Forward | TTACTTACAC | 139 | NM001024630 |
| | | CCCGCCAGTC | | |
| RUNX2 | Reverse | TATGGAGTGC | | |
| | | TGCTGGTCTG | | |

Cell Proliferation

Cell proliferation studies were performed by culturing osteoblasts in DMEM:F-12 complete growth medium for 1, 4 and 7 days. Cells on each substrate were detached using 1000 μL of trypsin, scraped off from the substrate, and pipetted into a preformed solution of 0.5 mL PBS and 0.3 mL Trypan blue for a total volume of 1.8 mL. Solution was mixed by pipetting, and then 15 μL of the homogenized solution was placed on a hemocytometer. Cells were counted under an inverted microscope, and a final number of cells in the total solution were determined. Cell counts were performed in triplicates.

Von Kossa Staining

Von Kossa staining can be used to determine the presence of mineralization by osteoblasts. First, cultured osteoblasts were rinsed in PBS, and subsequently fixed in 10% neutral buffered formalin (Sigma Aldrich, St. Louis, Mo.) solution for 10 min, and then rinsed 3 times in H₂O. Cells were then incubated in the dark for 30 min in 5% silver nitrate in H₂O, and exposed to ambient light for color development afterwards. Dark (black) staining in the cells is indicative of a positive stain for deposited mineral. Stains were observed using a Nikon Eclipse L150 optical microscope at 5× (five times) magnification.

Characterization of Substrates and Cultured Cells

Samples were imaged using a Philips XL30-FEG Scanning Electron Microscope (SEM) operated between 5 and 20 kV. Zeolite coated Ti6Al4V and substrates with cell culture were sputter coated with Au/Pd thin layer before SEM characterization.

Osteoblasts were fixed in 2.5% glutaraldehyde in 0.1 M sodium cacodylate solution for 1 hour at room temperature. Solution was pre-warmed to 37° C. Samples were then washed 3 times in 0.1 M sodium cacodylate, and incubated in mix solution of 1% osmium tetraoxide and 1% sodium cacodylate for 1 hour at room temperature. Samples were subsequently dehydrated in ethanol series (30%, 50%, 70%, 80%, 95%, and 100%) for 15 minutes each and critical point dried using Balzar's critical point dryer before sputter coating.

Statistical Analysis

Statistical analysis was performed using t-test method with p<0.05 as criteria for rejecting the null hypothesis.

Materials and Tissue Characterization

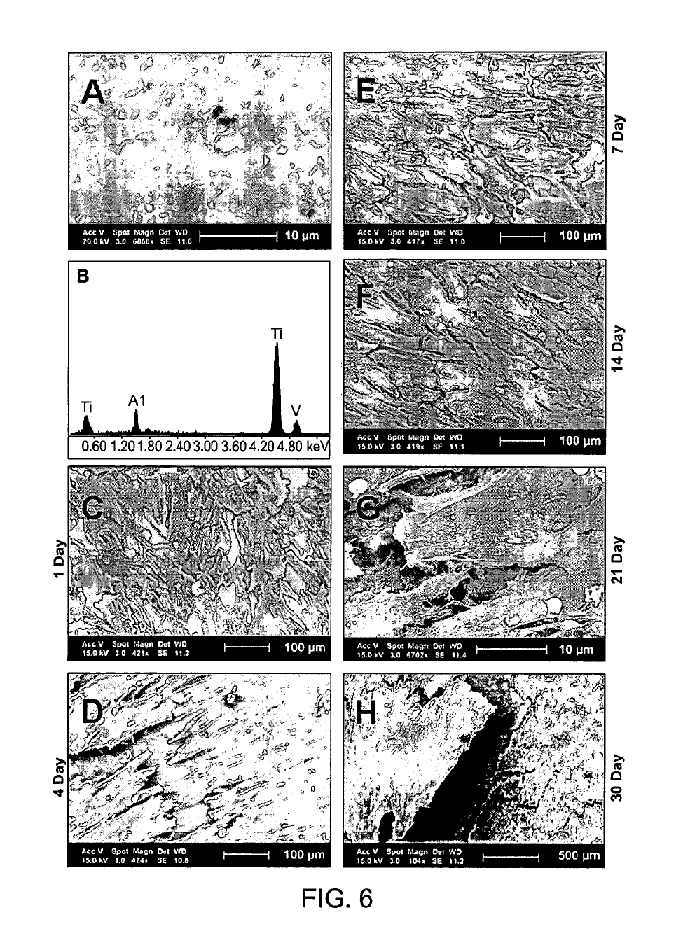

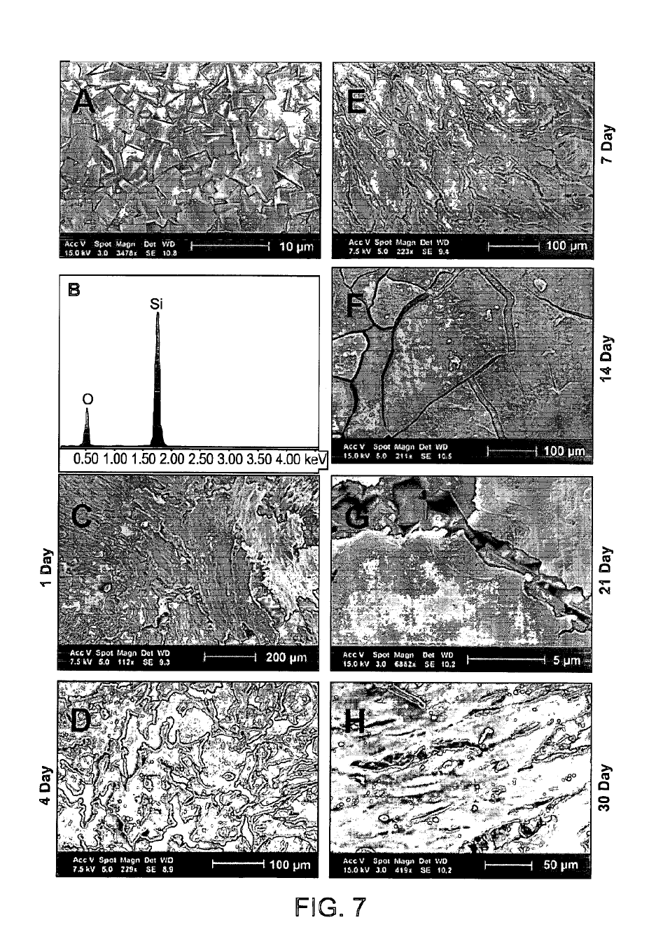

SEM micrograph of Ti6Al4V shows a highly smooth surface with very small roughness (FIG. 6A), and its chemical composition is confirmed with EDS (FIG. 6B). Uniform MFI coating was obtained on Ti6Al4V alloy (FIG. 7A) and its elemental composition was confirmed with EDS (FIG. 7B) as reported previously. Osteoblasts were cultured on bare and MFI-coated Ti6Al4V substrates for up to 30 days. SEM micrographs of cultured hFOBs on Ti6Al4V and MFI-coated Ti6Al4V show a greater number of osteoblasts, a higher degree of cell intergrowth, and a denser monolayer present on zeolite coatings as compared with bare Ti6Al4V substrates at the same culturing time.

A confluent layer of osteoblasts was observed on Ti6Al4V after 4 days of culture and more solid bone-like structure was observed in samples taken at 21 days. Osteoblasts did not adhere very well to Ti6Al4V substrates. Delamination of osteoblast layer was observed in samples taken after 4 days of culture, and it increased over time. Delamination of the whole cellular layer is seen in 30 day cultures on Ti6Al4V (FIG. 6H). Mineralization and formation of fibers were observed after 14 days of culture, and both are highly noticeable in 21 day cell culture samples (FIGS. 6F & 6G). Osteoblast cell morphology on titanium substrates was flat and only a few round nuclei were observed. Fewer cell to cell contacts were seen and three-dimensional cellular web network was lacking.

A confluent osteoblast layer (similar to bare alloy samples) was obtained on MFI coatings after 4 days of culture, and a hard bone-like structure covered the surface of the coating at 14 days (one week faster than Ti6Al4V). Mineralization nodules were also observed earlier (4 days) than on titanium substrates (14 days). Fibrous tissue was difficult to visualize at 4 days, but noticeable at 14 days of culture. Osteoblasts did not seem to delaminate from the zeolite coating surface even after 30 days of culture (FIGS. 7A-H). FIG. 7F shows a mineralized osteoblast layer that fractured after being subjected to high pressures during critical point drying process, yet it did not delaminate from the base MFI coating. Round osteoblast cell morphology was observed on MFI coatings with a higher number of round nuclei than titanium substrates. A highly interwoven extracellular matrix (ECM), comprised of fiber, mineral and cellular material, was observed on MFI samples. A three-dimensional cellular web-like network was observed with interconnecting cell junctions. Area around the nuclei had ridge like characteristics. Osteoblasts were seen bridging the zeolite intercrystalline gaps, and multilayered osteoblast tissue was observed.

Better osteointegration can result from the highly interwoven extracellular matrix (ECM) deposited by osteoblasts on a rough MFI surface. Osteoblasts exhibited round morphology with a high degree of cytoplasmic extensions (for intercellular communication and attachment to the surface), indicative of healthy cellular functions. In contrast, osteoblasts on Ti6Al4V had a flat morphology with fewer interconnecting junctions than on MFI coated substrates. It can be appreciated that the results were in agreement with previously published reports on osteoblast morphology on unaltered Ti6Al4V as well as sandblasted Ti₆Al₇Nb with a 3-D surface topology. Bare Ti6Al4V has a flat surface that does not provide structural support for osteoblasts. With a 3-D surface topology of zeolite crystals, osteoblasts have greater structural support and abundant sites for cell adhesion, which results in better cellular adhesion to zeolite surface than seen on bare Ti6Al4V. No delamination of tissue was observed MFI-coated Ti6Al4V, but significant peeling off of cellular layer was seen on bare Ti6Al4V.

Corrosion Resistance

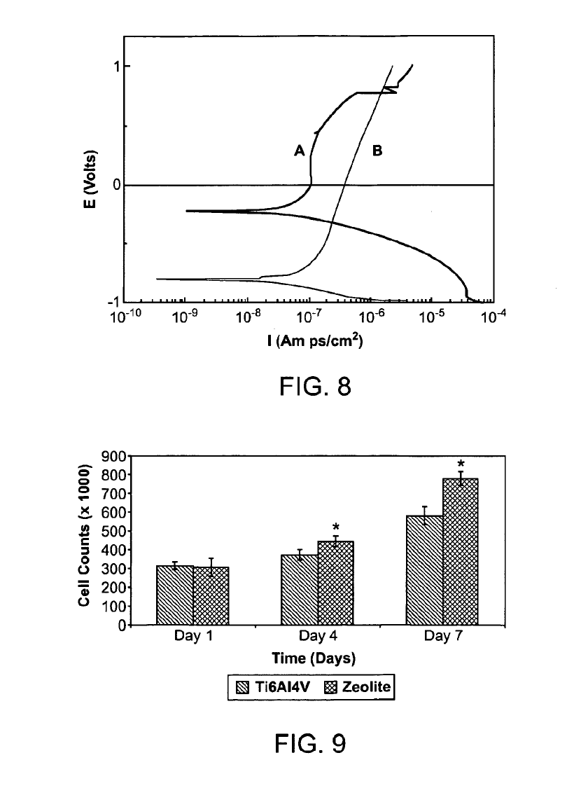

Corrosion resistance of MFI-coated and bare Ti6Al4V are shown in FIG. 8. MFI-coated Ti6Al4V substrates also show superior corrosion resistance in 1× PBS solution. A much lower corrosion potential and a lower final current density were observed for MFI-coated substrates than bare Ti6Al4V substrates (FIG. 8). Lower corrosion rates were calculated for MFI-coated substrates (1.7 E-11 mm per year) than bare Ti6Al4V (9.5 E-12 mm per year).

Superior corrosion resistance will prevent the titanium alloy implant from corroding and releasing toxic ions into the surrounding tissue. It can increase the lifespan of the implant and reduce the need for recurring surgeries for patients with metallic implants. Current implants last between 10 and 15 years, and if zeolite coatings can potentially double or triple the lifespan of the implant, most patients will never need a second surgery from implant failure due to corrosion.

Cell Proliferation and Von Kossa

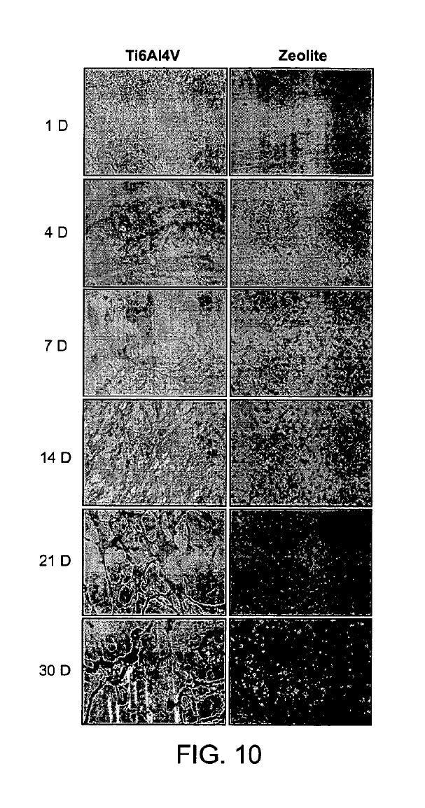

Osteoblast proliferation was measured on bare and MFI-coated Ti6Al4V using trypan blue staining for 7 days in DMEM:F-12 complete growth (non-mineralizing) media. The zeolite surface shows no difference in osteoblast proliferation after 1 day of culture, but 19% and 34% higher cell count than the bare alloy surface after 4 and 7 days of culture was seen, respectively (FIG. 9). Hydroxyapatite mineralization by osteoblasts was observed using Von Kossa staining, and appears as black nodules in the optical images (FIG. 10). A positive identification of hydroxyapatite mineral was obtained after 4 days of culture on MFI coated alloy and after 14 days on bare alloy. Furthermore, bare alloy was covered intermittently with mineral secreted by osteoblasts while MFI coated alloy shows a complete coverage after 21 days of culture (FIG. 10).

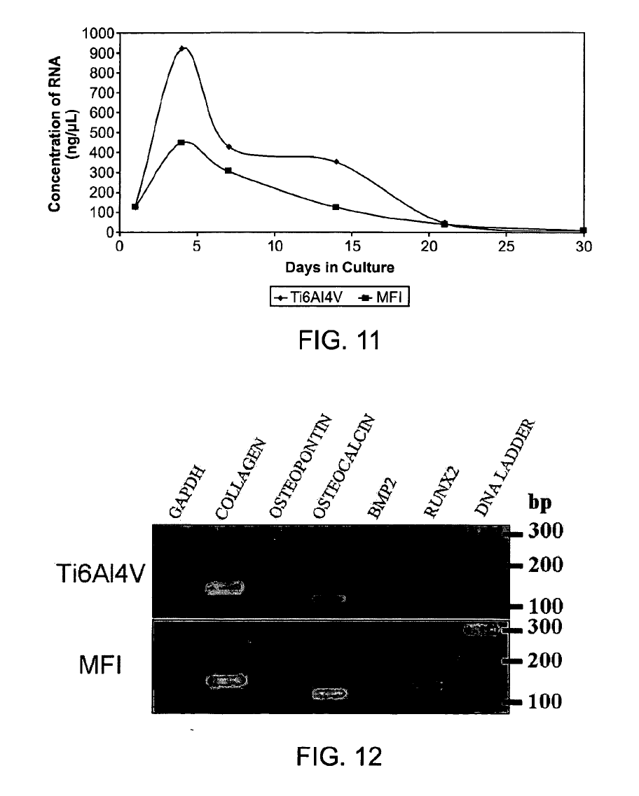

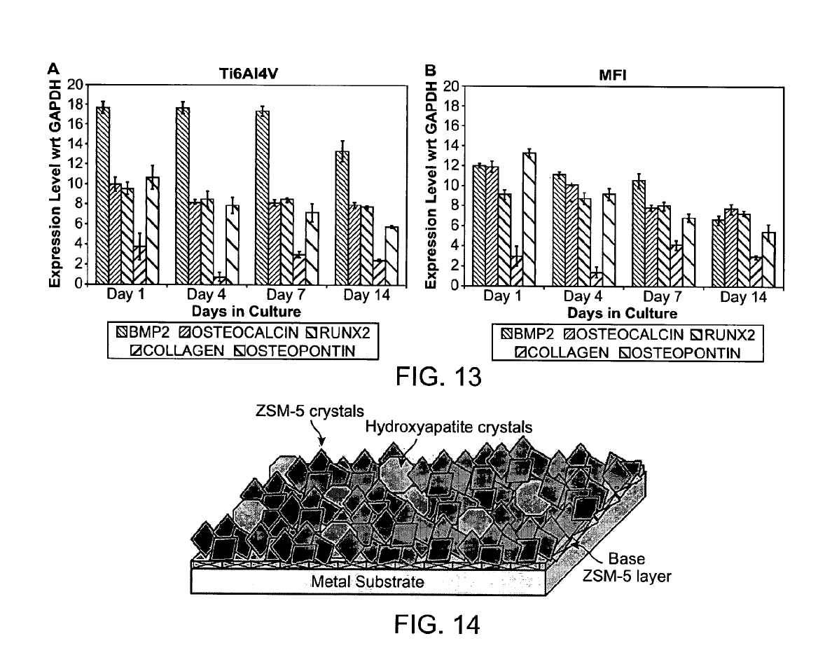

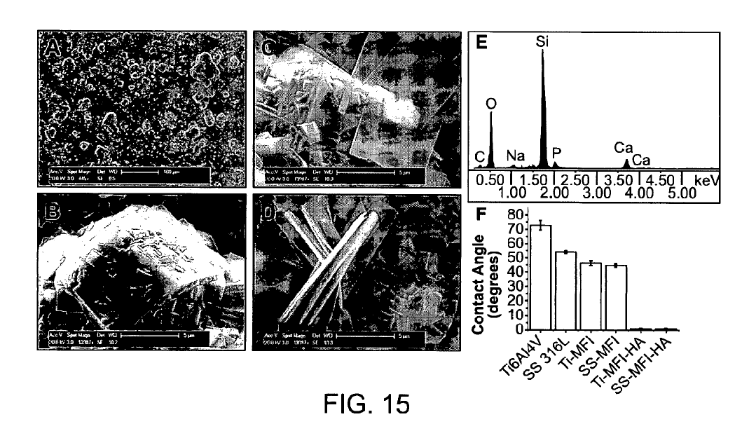

Higher proliferation of osteoblasts on MFI coated Ti6Al4V versus the bare alloy clearly indicates a difference in osteoconductivity of the two materials; MFI surface shows higher osteoconductivity than Ti6Al4V. Cells not only proliferate faster on MFI surface but also start mineralizing earlier. While mineralization by hFOBs takes 14 days on bare Ti6Al4V, it is seen in only after 4 days of culture on MFI-coated Ti6Al4V (FIG. 11). Significantly more black mineral nodules are observed on MFI-coated substrates than on bare Ti6Al4V, indicating a higher degree of mineralization on zeolite surface. Decrease in mineralization content on bare alloy from 21 days of culture to 30 days of culture can be explained by delamination of the cellular layer. This phenomenon was not observed on MFI coated alloy indicating superior cellular adhesion to MFI surface. In contrast, MFI surface appears completely black with evenly deposited mineral nodules.

These results can be attributed to a 3-D structure of zeolite crystals that provide a more natural bone like environment for cells. A microcrystalline environment with nanometer sized pores provides a nano-micro topology that is ideally suited for bone growth. Better adhesion and faster mineralization of osteoblasts on MFI surface can be explained by understanding the process of bone formation. Since mineralization succeeds cellular adhesion and ECM formation in osteogenesis, surface texture indirectly plays an important in mineralization time. A 3-D surface provides more binding sites for ECM proteins that anchor osteoblasts. Adhesion of cells is strengthened after the formation of a highly interwoven ECM on the MFI surface, which is immediately followed by mineralization. A short adhesion time will therefore translate into a shorter mineralization time as observed on MFI coated alloy versus the bare alloy. Furthermore, an interwoven ECM formation also prevents delamination of the cellular layer from the substrate's surface, which is highly desirable for enhancing osteointegration. Thus, MFI coated Ti6Al4V implants may be more osteointegrative than bare Ti6Al4V implants.

RNA Content and Gel Electrophoresis

To further quantify osteoconductive and osteoinductive properties of zeolite MFI surface on hFOBs, cellular gene expression was quantified. Firstly, it is imperative to check whether human fetal osteoblastic (hFOB) cells have differentiated into mature osteoblasts, and if they are performing their natural secretory functions. The expression of specific genes, such as BMP2, GAPDH, Osteocalcin, RUNX2, ColT1Al, and Osteopontin, indicates the differentiation of hFOBs to mature osteoblasts (Table 2). Expression of these genes was tested by analyzing RNA from cultured cells over time.

Concentrations of RNA isolated from osteoblast cell cultures on bare and MFI-coated Ti6Al4V coupons increased up to 4 days of culture and then steadily decreased until virtually no RNA was obtained from cells at 30 days of culture. The concentration of RNA from osteoblasts cultured on MFI-coated Ti6Al4V was lower than from cells cultured on bare Ti6Al4V. RNA concentration from osteoblasts cultured on both substrates started out equal after 1 day of culture, deviated from day 4 to day 14, and the equalized again at 21 days of culture. Furthermore, the presence of test genes was confirmed by gel electrophoresis. Bands for all genes were visible at the appropriate base pair lengths as indicated in Table 3.

Osteoblasts were cultured in non-mineralizing complete basic media at 34° C. for the first two days, which is suitable for proliferation of osteoblasts. Osteoblasts were not mineralizing while they continued to divide and grow in number. Hence, RNA obtained from day 1 was equivalent in osteoblasts cultured on bare and MFI-coated substrates. After cells were transferred to osteogenic media, hFOBs started to differentiate into adult osteoblasts and mineralize. Mineralization of cells reduces the number of cells is the first step in the formation of hard bone tissue, and osteoblasts cease to function after they enclose themselves in hydroxyapatite mineral. Furthermore, RNA cannot be extracted from osteoblasts by methods used without demineralization. From this evidence it can be appreciated that more mineral was being deposited by osteoblasts on MFI-coated substrates than on bare Ti6Al4V substrates. The number of cells originally seeded and the size of the substrates was kept constant to avoid confounding of results. The results confirm that MFI coatings induce differentiation faster in osteoblasts than bare Ti6Al4V. It can be appreciated that the 3-D microstructure of zeolite crystals provide a large surface area for proteins to bind and anchor the cells onto the surface so that osteoblasts can start mineralizing faster on zeolite surface than on Ti6Al4V.

RT-QPCR

Collagen TlAl was the most highly expressed gene, while BMP2 was the least expressed gene, while Osteopontin, Osteocalcin, and RUNX2 were almost equally expressed. These trends were valid at different osteoblast culture times and for different culturing substrates. A 6-fold increase in BMP-2 expression was seen in osteoblasts cultured on MFI-coated Ti6Al4V versus bare Ti6Al4V. Collagen and RUNX-2 expression did not differ with substrates. However, Osteopontin and Osteocalcin expressions were slightly lower on zeolite for day 1 and day 4 cultures, but showed higher level of expression after seven days of culture.

BMP-2 has been shown to be involved in osteogenic transformation of bone cells. Here, a six-fold increase in the expression of BMP-2 on MFI-coated Ti6Al4V versus bare substrates is shown, which indicates that zeolite coatings have osteoinductive effect on hFOBs. This correlates well with Von Kossa results which indicate that osteoblasts started mineralizing seven days faster on zeolite coatings than on bare Ti6Al4V substrates. Collagen and RUNX-2 did not show any significant difference in expression levels, which indicate that cellular functions responsible for depositing an extracellular matrix are comparable in osteoblasts cultured on both substrates.

However, expression of Osteocalcin and Osteopontin genes show interesting anomalies. Up to four days in culture, a lower expression is observed in osteoblasts on zeolite coated substrates, while a higher expression is observed on zeolite surfaces beyond day 4. It can be appreciated that higher osteoconductivity of zeolite surface is responsible for greater osteoblast proliferation for the first four days in culture, and explains a lower osteoblast mineralization rate for the first four days. Once a complete formation of extracellular matrix has occurred, osteoblasts start mineralizing and an increase in Osteocalcin and Osteopontin genes is observed in cells cultured on zeolite surfaces. This evidence points to a higher osteoconductivity as well as a higher osteoinductivity of zeolite surface as compared to bare Ti6Al4V surfaces.

It can be appreciated that as shown above, zeolite MFI coatings can be successfully applied to Ti6Al4V substrates and shown to be highly corrosion resistant in 1× PBS media, and biocompatible. hFOBs proliferated and differentiated better on MFI coatings than on bare titanium alloy substrates. Higher cell count was seen on zeolite coatings versus Ti6Al4V at similar culturing times. A six-fold increase in BMP-2 was observed in osteoblasts cultured on MFI surface than on Ti6Al4V surface, indicating the osteoinductive effect of MFI surface. Higher levels of Osteocalcin and Osteopontin were seen which refer to enhanced differentiation of osteoblasts on the MFI surface. SEM and Von Kossa results confirmed that tissue layer did not flake off from the zeolite surface after 30 days while tissue delaminated from the titanium alloy surface even at 14 days. It can be appreciated that MFI coatings on Ti6Al4V can enhance the osteointegration of orthopedic and dental implants while increasing their overall lifespan by obstructing corrosion processes, and which can lead to fewer recurring surgeries for patients and improve healthcare for millions worldwide.

Synthesis and Characterization of MFI-HA Composite Coating

In accordance with another exemplary embodiment, a three step synthesis procedure was used to synthesize MFI-HA coatings on Ti6Al4V substrates. First, an MFI coating was deposited directly on the metal substrate as shown previously. In a second step, the MFI coated substrate is dipped into a homogenized 2 grams per 5 mL ethanol solution. The coating is dried in air for 10 minutes followed by a 15 minute drying period in a convection oven to ensure complete removal of ethanol form the hydroxyapatite layer. Finally, a 4 hour short MFI synthesis is carried out to bind the hydroxyapatite crystals to the base MFI layer. Characterization of the coating was carried out using an X-ray diffractometer, and a Scanning Electron Microscope (SEM) equipped with an EDS (Energy dispersive X-ray spectroscopy) system. A DC Polarization method was used to determine the corrosion resistance of the coating and bare Ti6Al4V. The coating was polished and nano-indentation was used to determine the hardness and modulus of the coating. Hydrophilicity of the coating was measured using the Optima contact angle system.

As shown in FIGS. 15A-15D, a layered zeolite based composite coating was formed on a titanium alloy, Ti6Al4V. In accordance with an exemplary embodiment, the coating exhibits a mixed nano-micro crystalline structure. It can be appreciated that in accordance with an exemplary embodiment, nano-hydroxyapatite crystals of less than 200 nm diameters were used in conjunction with the 2-5 μm size MFI crystals formed by in situ hydrothermal synthesis. It can be appreciated that elemental analysis confirmed the presence of major components of hydroxyapatite, Ca and P, on the coating's surface (FIG. 15E). In accordance with an exemplary embodiment, a few needle-like crystals were observed after 4 hour MFI synthesis, which can be due to slight recrystallization of hydroxyapatite in MFI synthesis solution. In accordance with an exemplary embodiment, the zeolite crystals “locked-in” the hydroxyapatite crystals while still allowing access to the mineral from the surface.

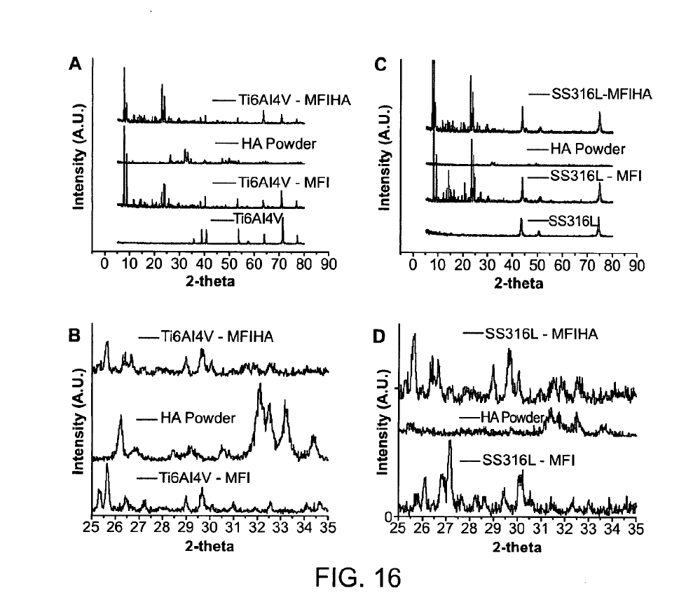

In accordance with another exemplary embodiment, the presence of hydroxyapatite further increased the hydrophilicity of the coating as compared to Ti6Al4V and bare MFI coatings (FIG. 15F), and contact angle was measured to be below 1 degrees. It can be appreciated that a more hydrophilic surface is widely accepted to provide better cellular adhesion for osteoblasts during bone formation, and crystalline hydroxyapatite surface is desired for faster osteoblast proliferation and differentiation, which is important for stimulating the bone-healing process. An added advantage is that zeolite microtopology provides a large surface area for cell adhesion, and nano-hydroxyapatite crystals will provide anchor points for the extracellular matrix and the mineral secreted by ostebolasts during bone formation. In accordance with an exemplary embodiment, x-ray diffraction patterns confirmed the presence of hydroxyapatite after the final 4 hour MFI synthesis step indicating there was no loss of crystalline structure (FIGS. 16A-D) of the powder after in situ MFI crystallization.

### Corrosion Resistance and Mechanical Properties

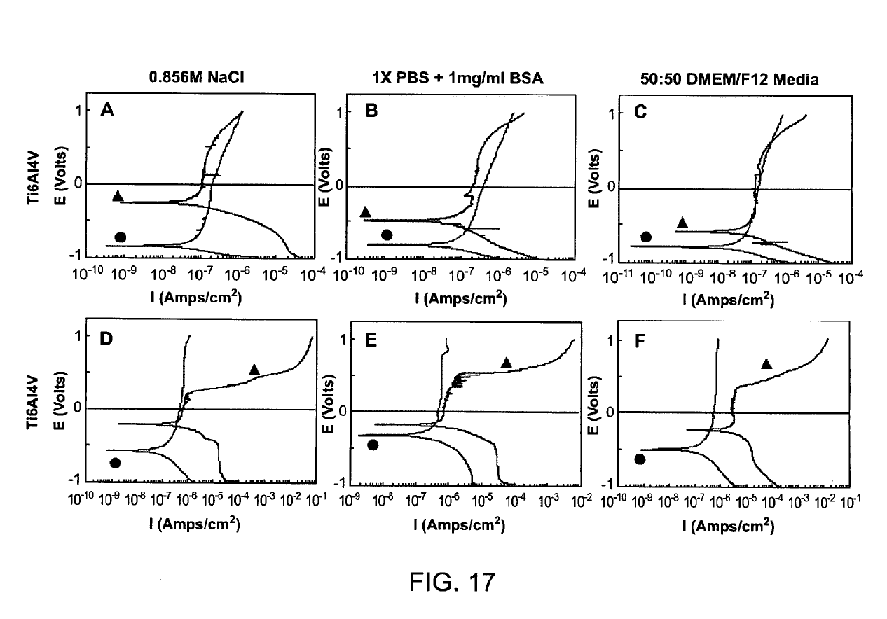

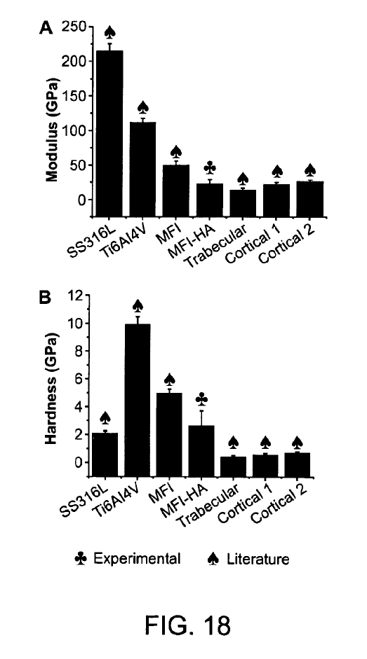

In accordance with another exemplary embodiment, two major functions of the MFI-HA composite coating are: (1) to protect the metallic implant from corroding and releasing toxic ions into the surrounding tissue, and (2) to mimic bone properties and eliminate the modulus mismatch between the implant and bone. MFI-HA coated Ti6Al4V showed slower corrosion rates (data not shown) than bare Ti6Al4V in 0.856 M NaCl, 1× PBS solution with 1 mg/ml BSA, and 1:1 DMEM/F-12 media. MFI-HA coated Ti6Al4V always had a lower open circuit potential, and a lower final current density indicating better corrosion resistance than bare Ti6Al4V (FIGS. 17A-17C). Corrosion resistance of Ti6Al4V decreases with increasing biological complexity of the media, while MFI-HA coating shows no change in corrosion resistance. DMEM media includes several active proteins that simulate in vivo conditions, and show that MFI-HA coating can withstand a biologically active corrosive environment better than titanium alloy. It can be appreciated that as previously shown, the enhancement in corrosion resistance provided by the base MFI coating also prevents the leaching of any toxic Al and V ions into the solution.

It can be appreciated that in accordance with another exemplary embodiment, in addition to corrosion protection, MFI-HA coating possesses a modulus and hardness that mimics those of natural human bone. The elastic modulus is an indication of resistance to elastic or recoverable deformation and hardness is an indication of resistance to plastic or permanent deformation. It can be appreciated that a mismatch between the modulus of Ti6Al4V (110 GPa) and bone (13-26 GPa) can cause bones to crack and loosen from the implant over time, leading to poor osteointegration. MFI coatings deposited on metallic alloys show a modulus which is half (40-50 GPa) that of Ti6Al4V, but still not comparable to that of bone. As shown in FIG. 16D, MFI-HA coating on Ti6Al4V alloys possess a modulus of 23.3 ±5.8 (s.d.) GPa. Although hardness of the MFI-HA coating is not critical to implant function, a reduction in hardness from 5 GPa (MFI) to 2.7±1.0 (s.d.) GPa (MFI-HA) was observed (FIG. 17E). This is due to a mixed MFI and hydroxyapatite layer that is exhibiting average properties of both inorganic materials. Hydroxyapatite is a much softer material than zeolite, and this is apparent in the hardness of MFI-HA composite coating. Matching the modulus of bone will prevent implant loosening and enhance osteointegration, leading to a longer implant lifespan. This is important to prevent recurring surgeries for implant replacement, thus improving human health and reducing the cost of healthcare for orthopedic patients.

In accordance with an exemplary embodiment, a novel synthesis method of a zeolite based MFI-HA biocompatible coating for improving osteointegration of metallic implants is disclosed. The composite coating is superhydrophilic and hydroxyapatite is accessible at surface for inducing osteoblast proliferation and differentiation. Zeolite crystals that glue the hydroxyapatite on to the base MFI coating further increase the surface area of the coating, and will provide increased number of attachment points for osteoblast cells. It can be appreciated that a MFI-HA coating is highly corrosion resistant in aggressive pitting NaCl media, phosphate buffer solution with BSA protein, as well as highly complex DMEM cell culture media, and outperforms the state-of-the-art Ti6Al4V that are widely used for orthopedic applications. Mechanical properties of MFI-HA coating mimic those of natural bone, and completely eliminate the modulus mismatch problem that researchers have been trying to solve for the past 40 years. The MFI-HA coating system is robust; the base MFI layer can be synthesized on various metals and alloys thus potentially eliminating the need for using expensive titanium as the metal of choice for orthopedic implants.

It can be appreciated that in accordance with an exemplary embodiment, the commercialization of MFI-HA coating can vastly improve human health in older age while dramatically cutting healthcare costs of procedures such as total hip arthroplasty. For example, reducing the cost of a $6000 implant by $1000 could save up to $600 million on over 600,000 implants annually. This can be achieved by using cheaper stainless steel implants instead of more expensive titanium implants. Patients can also expect increased implant lifespan and durability, and faster post-surgical recovery due to increased osteointegration.

### Synthesis

In accordance with an exemplary embodiment, a three step synthesis procedure was used to synthesize MFI-HA coatings on Ti6Al4V and SS316L substrates that are routinely used for orthopedic implants.

Step 1: Formulation of Base MFI Coating on Metal Surface

High-silica-zeolite (HSZ) MFI coatings were prepared by an in-situ hydrothermal crystallization method. First, a clear synthesis solution with molar composition 0.16TPAOH:0.64NaOH:TEOS:92H₂O:0.0018Al (weight compositions: 17.03 g TPAOH, 5.36 g NaOH, 43.60 g TEOS, 336.00 g H₂O, 0.0105 g Al) was prepared by dissolving aluminum powder (200 mesh, 99.95+ wt %, Sigma Aldrich, St. Louis, Mo.) in sodium hydroxide (99.99 wt %, Sigma Aldrich, St. Louis, Mo.) and de-ionized (DI) H₂O followed by drop-wise addition of tetrapropylammonium hydroxide (TPAOH, 40 wt %, aqueous solution, SACHEM, Austin, Tex.) and tetraethylorthosilicate (TEOS, 98 wt %, Sigma-Aldrich, St. Louis, Mo.) under stirring. The clear solution was aged at room temperature for about 4 hours under stirring before use.

A 2 L Teflon-lined Parr (Moline, Ill.) autoclave was used as the synthesis vessel and the substrate was suspended vertically inside the synthesis solution using a Teflon® holder and steel wire. Crystallization was carried out in a convection oven at 175° C. for 24 hours. The autoclave was then removed and quenched with tap water. The coated sample was rinsed with DI H₂O and dried in ambient room air.

Step 2: Formation of Hydroxyapatite Layer by Dip-Coating

The MFI coated substrates were cut into smaller coupons (1 inch×1.5 inch). A hydroxyapatite (97%, <200 nm, Sigma Aldrich, St. Louis, Mo.) suspension was prepared in ethanol (EtOH, 100%, Sigma Aldrich, St. Louis, Mo.) by adding 2 grams of hydroxyapatite powder to 5 mL of ethanol. The suspension was homogenized by vortexing for 15 seconds. MFI coated Ti6Al4V and SS 316L coupons were subsequently dipped into a homogenized HA-EtOH suspension and excess solution was gently drained by dabbing the panel on to a paper towel. Dip-coated panels were air dried for 10 minutes and then dried in a convection oven at 60° C. for 15 minutes to completely evaporate the ethanol from the hydroxyapatite layer.

Step 3: Interlocking HA Crystals with a Short MFI Synthesis

A subsequent short 4 hour MFI synthesis was carried out on top of the n-HA layer to lock in the HA particles within the zeolite structure and bind the HA to the base MFI layer. Same solution formulation as in step 1 was used for MFI synthesis, which was carried out in a Teflon lined 45 mL autoclave (Parr Instrument Co., Moline, Ill.) at 175° C. for 4 hours.

Cell Culture and Gene Expression

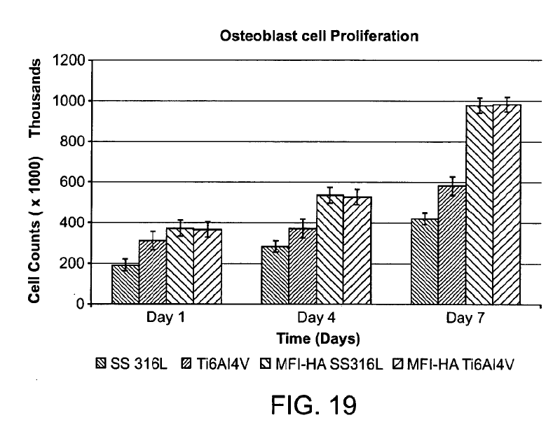

In accordance with another example, the biocompatibility of the composite MFI-HA coating was tested and compared to bare Ti6Al4V and SS316L substrates. Human fetal osteoblast cells were cultured on bare and MFI-HA coated Ti6Al4V and SS316L substrates for 30 days. Cell proliferation assay showed a higher cell count on MFI-HA coated substrates as compared to bare substrates (FIG. 19). Osteoblast proliferation was measured using trypan blue staining for 7 days in DMEM:F-12 complete growth (non-mineralizing) media. After 24 hrs of cell culture, coated surfaces show higher osteoblast cell counts than bare SS316L substrate but comparable to the bare Ti6Al4V substrate. Day 4 shows significantly higher cell counts on the coated substrates as compared to both uncoated substrates, and the difference between the coated and uncoated substrates increases further by day 7. No difference in cell counts was observed between MFI-HA coated SS316L and Ti6A4V substrates indicating that the coating surface did not significantly differ in biocompatibility on different substrates. Higher cell proliferation of osteoblasts on the MFI-HA surfaces indicates a higher bioactivity of the surface, which may be responsible for the osteoconductive effect on hFOBs. It is known that hydroxyapatite is highly bioactive, and is the major component of bone, and we have previously shown zeolites to have an osteoconductive and osteoinductive effect on hFOBs.

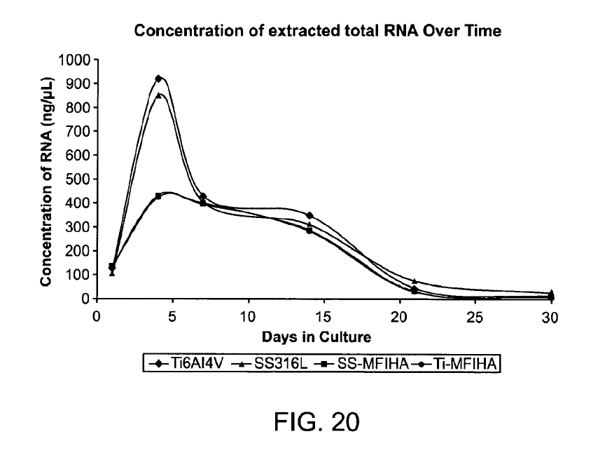

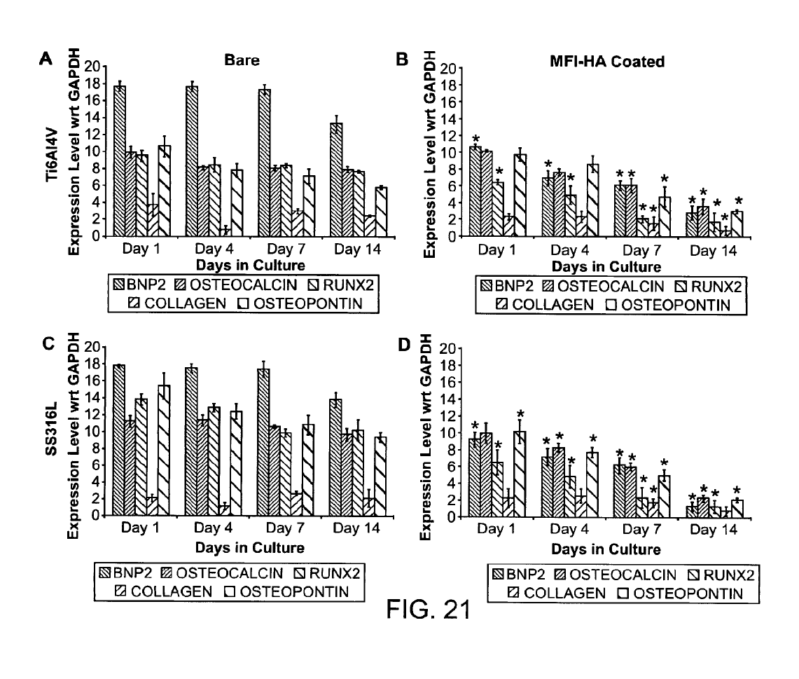

Gene expression of hFOBs was quantified over 30 days of culture to verify osteoblast differentiation into a fully mature phenotype. Total RNA content was analyzed before performing RT-qPCR (FIG. 20). RNA concentrations from osteoblast cell cultures on bare Ti6Al4V and SS316L substrates were the same as those obtained from cells on coated substrates after 1 day of culture, but were much higher for day 7, and equalized again after day 7. Very small quantity of RNA was obtained after 21 and 30 days of culture, and was not enough to perform RT-qPCR analysis. Osteoblasts were cultured in non-mineralizing complete basic media at 34° C. for the first two days, which is suitable for cell growth and not mineralization. RNA obtained after 1 day of culture was equivalent in osteoblasts cultured on bare and MFI-HA coated substrates. The elevated RNA content in osteoblasts cultured on uncoated substrates can be explained by decreased mineralization activity of osteoblasts when switched to the osteogenic mineralization media after 2 days of culture. Culturing osteoblasts on a bioactive surface has an osteoinductive effect on the cells and osteoblasts start to mineralize faster than on a non-bioactive surface. Once mineralization occurs, cells get entrapped in the mineralized matrix, and it is difficult to remove RNA from the cells without using harsh demineralization steps, thereby decreasing the concentration of total RNA that can be extracted. This evidences points to the higher osteoinductive effect of MFI-HA coating surface on hFOBs.

To determine if hFOBs were differentiating into adult osteoblasts, the expression of specific genes, such as Bone morphogenetic protein-2 (BMP2)³⁹, Glyceraldehyde 3-phosphate dehydrogenase (GAPDH), Osteocalcin, Runt-related transcription factor-2 (RUNX2), Collagen Type 1 Alpha 1 (ColTlAl), and Osteopontin was analyzed. GAPDH is used as a housekeeping gene since it is responsible for performing necessary metabolic and other cellular functions. Collagen expression is related to structural support and extracellular matrix formation. Osteocalcin expression is important for bone metabolism and hydroxyapatite formation, while Osteopontin binds to hydroxyapatite and is important anchoring bone cells to the extracellular matrix. BMP2 induces osteogenic transformation in hFOBs, and RUNX2 has been shown to be the principal osteogenic master switch.

Collagen TlAl was expressed the most, while BMP2 was the least expressed gene. Osteopontin, Osteocalcin, and RUNX2 were almost equally expressed, and these trends were valid for all hFOB culture samples taken over two weeks. These trends were also unaffected by the difference in substrates, although the overall gene expression levels varied significantly. An eight-fold increase in BMP-2 expression was seen in osteoblasts cultured on MFI-HA coated SS316L versus bare SS316L, and a six-fold increase was observed on MFI-HA coated Ti6Al4V versus the bare metal. RUNX-2 gene expression saw a comparable change on both Ti6Al4V and SS316L substrates when covered with MFI-HA composite coating. Collagen, Osteopontin, and Osteocalcin gene expression did not show and increase on MFI-HA coated substrates as compared to the bare substrates until after 7 days of culture. No significant difference in hFOB gene expression was observed on MFI-HA coated SS316L and Ti6Al4V indicating the lack of interference by the type of metal used.

BMP-2 has been shown to be involved in osteogenic transformation of bone cells. Here, a six and eight fold increase in the expression of BMP-2 on MFI-coated Ti6Al4V and SS316L, respectively versus the corresponding bare substrates is shown, which indicates that the composite coatings has an osteoinductive effect on hFOBs. Higher RUNX-2 levels after 7 days in culture suggests that more hFOBs started mineralizing thereby leading for faster mineral deposition. In the human body, this can lead to faster osteointegration of implants, and faster patient recovery from bone and joint replacement surgeries. Furthermore, MFI-HA coated Ti6Al4V and SS316L show no difference in performance in vitro, and their efficacy in vivo should be tested to determine if expensive Ti6Al4V materials can be replaced with cheaper SS316L to make orthopedic implants more affordable in developing countries.

It will be understood that the foregoing description is of the preferred embodiments, and is, therefore, merely representative of the article and methods of manufacturing the same. It can be appreciated that many variations and modifications of the different embodiments in light of the above teachings will be readily apparent to those skilled in the art. Accordingly, the exemplary embodiments, as well as alternative embodiments, may be made without departing from the spirit and scope of the articles and methods as set forth in the attached claims.

What is claimed is:

1-45. (canceled)

46. A composite comprising:
a base zeolite layer comprising a first plurality of zeolite crystals; and
a hydroxyapatite layer disposed on the base zeolite layer, wherein the hydroxyapatite layer comprises a plurality of hydroxyapatite crystals and a second plurality of zeolite crystals, wherein the plurality of hydroxyapatite crystals is locked in the second plurality of zeolite crystals to form an interlocked structure.

47. The composite of claim 46, wherein the base zeolite layer or the hydroxyapatite layer is prepared by an in-situ hydrothermal crystallization.

48. The composite of claim 46, wherein the base zeolite layer is formed by combining a silica source with a zeolite-forming structure directing agent.

49. The composite of claim 48, wherein the silica source is an organic silicate.

50. The composite of claim 48, wherein the silica source is tetraethyl orthosilicate, tetramethyl orthosilicate, fumed silica, silica gel or collodial silica.

51. The composite of claim 48, wherein the structure directing agent is an organic hydroxide.

52. The composite of claim 51, wherein the structure directing agent is tetrapropylammonium hydroxide, tetraethylammonium hydroxide, triethyl-n-propyl ammonium hydroxide or benzyltrimethylammonium hydroxide.

53. The composite of claim 46, wherein the first plurality of zeolite crystals is an aluminosilicate having a silicon to aluminum ratio of at least 5 to 1.

54. The composite of claim 46, wherein the hydroxyapatite layer is prepared using a colloidal hydroxyapatite suspension.

55. The composite of claim 54, wherein the collodial hydroxyapatite suspension comprises a mixture of hydroxyapatite powder and ethanol.

56. A composition comprising:
a substrate comprising a metal; and
a composite layer comprising a composite of claim 46, wherein the composite layer is disposed on the surface of said substrate.

57. The composition of claim 56, wherein the metal is titanium, steel, nickel or alloys or mixtures thereof.

58. The composition of claim 56, wherein the substrate is used for bioimplants.

59. A biocompatible implant comprising a substrate comprising a metal; and
a composite layer comprising a composite of claim 46, wherein the composite layer is disposed on the surface of said substrate.

60. The biocompatible implant of claim 59, wherein the implant comprises a dental implant, a cardiovascular implant or prosthesis.

61. A biocompatible implant comprising a substrate comprising a metal; and
a zeolite layer comprising a pure or high silica zeolite, wherein the zeolite layer is disposed on the surface of side substrate.

62. A method for preparing the composite of claim 46, said method comprising:
forming a base zeolite layer comprising a first plurality of zeolite crystals;
forming a hydroxyapatite layer on the base zeolite layer, wherein the hydroxyapatite layer comprises a plurality of hydroxyapatite crystals and a second plurality of zeolite crystals; and
interlocking the plurality of hydroxyapatite and the second plurality of zeolite crystals to form an interlocked structure.

63. The method of claim 62, wherein the base zeolite layer or the hydroxyapatite layer is prepared by an in-situ hydrothermal crystallization.

64. The method of claim 62, wherein the base zeolite layer is formed by combining a silica source with a zeolite-forming structure directing agent

65. The method of claim 62, wherein the silica source is an organic silicate.

66. The method of claim 62, wherein the silica source is tetraethyl orthosilicate, tetramethyl orthosilicate, fumed silica, silica gel or collodial silica.

67. The method of claim 62, wherein the structure directing agent is an organic hydroxide.

68. The method of claim 67, wherein the structure directing agent is tetrapropylammonium hydroxide, tetraethylammonium hydroxide, triethyl-n-propyl ammonium hydroxide or benzyltrimethylammonium hydroxide

69. The method of claim 62, wherein the base zeolite layer comprises a high-silica MFI zeolite or an aluminosilicate having a silicon to aluminum ratio of at least 5 to 1.

70. The method of claim 62, wherein the hydroxyapatite layer is prepared using a colloidal hydroxyapatite suspension.

71. The method of claim 70, wherein the collodial hydroxyapatite suspension comprises a mixture of hydroxyapatite powder and ethanol.

72. The method of claim 62, wherein the interlocking comprises an in-situ hydrothermal crystallizing the second plurality of zeolite crystals.

73. The method of claim 72, wherein the in-situ hydrothermal crystallizaing is shorter in length of time than the formation of the base zeolite layer.

74. The method of claim 62, wherein the step of forming hydroxyapatite layer comprising dip-coating.

75. The method of claim 62, further comprising forming the base zeolite layer on a substrate.

76. The method of claim 75, wherein the substrate is a metal used for bioimplants.

77. The method of claim 76, wherein the metal is titanium, steel, nickel or alloys or mixtures thereof.

78. A method for growing a cell on a substrate, said method comprising:
forming a zeolite coating on a substrate; and
proliferating a cell on the substrate.

79. The method of claim 78, wherein the zeolite coating comprises a base zeolite layer comprising a first plurality of zeolite crystals; and
a hydroxyapatite layer disposed on the base zeolite layer, wherein the hydroxyapatite layer comprises a plurality of hydroxyapatite crystals and a second plurality of zeolite crystals, wherein the plurality of hydroxyapatite crystals is locked in the second plurality of zeolite crystals to form an interlocked structure.

80. The method of claim 79, wherein the substrate comprises a metal selected from titanium, steel, nickel or alloys or mixtures thereof.

81. The method of claim 78, wherein said cell is a fibroblast, an embryonic stem cell or an osteoblast.

82. A method for inducing osteoblasts on a substrate or improving osteointegration of a substrate, said method comprising:
forming a zeolite coating on a substrate; and
proliferating osteoblasts on the substrate.

83. The method of claim 82, wherein the zeolite coating comprises a base zeolite layer comprising a first plurality of zeolite crystals; and
a hydroxyapatite layer disposed on the base zeolite layer, wherein the hydroxyapatite layer comprises a plurality of hydroxyapatite crystals and a second plurality of zeolite crystals, wherein the plurality of hydroxyapatite crystals is locked in the second plurality of zeolite crystals to form an interlocked structure.

84. The method of claim 82, wherein the substrate comprises a metal selected from titanium, steel, nickel or alloys and mixtures thereof.

85. The method of claim 82, wherein the substrate is a metallic implant.
